(19) United States
(12) Patent Application Publication
(10) Pub. No.: US 2008/0227787 A1
(43) Pub. Date: Sep. 18, 2008

(54) Use of New Lipoxygenase Inhibitors

(76) Inventors: Kristofer Olofsson, Stockholm (SE); Benjamin Pelcman, Stockholm (SE); Peter Nilsson, Stockholm (SE); Anders Hallberg, Uppsala (SE); Wesley Schaal, Stockholm (SE)

Correspondence Address: NIXON & VANDERHYE, PC, 901 NORTH GLEBE ROAD, 11TH FLOOR, ARLINGTON, VA, 22203, US

(21) Appl. No.: 10/590,450

(22) PCT Filed: Mar. 2, 2005
(86) PCT No.: PCT/GB2005/000780

§ 371 Date: Nov. 27, 2006

Related U.S. Application Data
(60) Provisional application No. 60/549,143, filed on Mar. 3, 2004.

(51) Int. Cl. A61K 31/16; A61K 31/381; A61K 31/44; A61K 31/415; A61K 31/498; C07D 241/36; C07D 213/78; C07D 231/10; C07C 243/16; A61P 29/00

(52) U.S. Cl. 514/249; 514/614; 514/438; 514/277; 514/406; 544/353; 546/314; 548/373.1; 564/250

(57) ABSTRACT

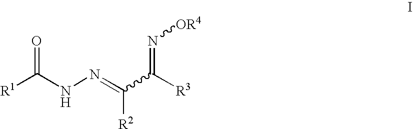

There is provided a use of a compound of formula (I), wherein R¹, R², R³ and R⁴ have meanings given in the description, and pharmaceutically-acceptable salts thereof, for the manufacture of a medicament for the treatment of a disease in which inhibition of the activity of a lipoxygenase (e.g. 15-lipoxygenase) is desired and/or required, and particularly in the treatment of inflammation.

### FIELD OF THE INVENTION

This invention relates to a novel use of certain compounds, some of which compounds are themselves novel and some of which are known. In particular, the invention relates to the use of such compounds in the inhibition of the activity of lipoxygenases, such as 15-lipoxygenase, and thus in the treatment of inflammatory diseases and of inflammation generally. The invention also relates to new compounds that are useful in that inhibition, to the use of such compounds as medicaments, to pharmaceutical compositions containing them, and to synthetic routes for their production.

### BACKGROUND

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomotology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas). Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

### PRIOR ART

Certain 1-aryl-2-(hydroxyimino)ethylidene arylhydrazides have been disclosed as being of potential use as antimicrobial and/or antibacterial agents in various prior art documents, including: Agarwal et al, Asian Journal of Chemistry, 2002 14, 489-492 and Ultra Scientist of Physical Sciences, 2001, 13, 267-270, Bahadur et al, Journal of the Indian Chemical Society, 1975, 52, 843-846, Misra et al, Indian Journal of Applied Chemistry, 1969, 32, 373-376, Varma et al, Indian Journal of Microbiology, 1964, 4, 63-66, Misra et al, Journal of the Indian Chemical Society, 1962, 39, 763-764, and Giammanco et al, Annali di Chimica, 1961, 51, 777-784 and ibid. 1961, 51, 175-179.

Other such compounds are disclosed in Agarwal et al, Asian Journal of Chemistry, 2000, 12, 843-846 and Atkinson et al, Journal of the Chemical Society, Abstracts, 1962, 1805-1811 as chemical curiosities and/or process intermediates. Other such compounds are commercially available, but (as far as the applicant is aware) have no perceived utility ascribed to them.

The use of the compounds disclosed in the above-mentioned documents in the treatment of disorders in which inhibition of the activity of lipoxygenases is required, and/or the treatment of inflammation generally, is neither mentioned nor suggested therein.

### DISCLOSURE OF THE INVENTION

According to the invention there is provided a use of a compound of formula I,

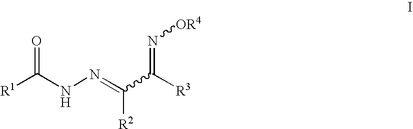

wherein
the squiggly bonds represent optional E or Z geometry;
R¹ and R² independently represent an aryl group or a heteroaryl group, both of which groups are optionally substituted by one or more substituents selected from:
X¹, C₁₋₈ alkyl, an aryl group and a heterocyclic group:—
(A) which C₁₋₈ alkyl group is itself optionally substituted by one or more Z substituents; and
(B) which C₁₋₈ alkyl, aryl and heterocyclic groups may themselves be substituted by one or more substituents selected from X¹, C₁₋₈ alkyl (which latter group may be further substituted by one or more substituents selected from X¹, C₁₋₈ alkyl, an aryl group, a heterocyclic group and Z), an aryl group and a heterocyclic group (and which latter two groups may be further substituted by one or more substituents selected from X¹, C₁₋₈ alkyl, an aryl group and a heterocyclic group), in which:—
X¹ represents, on each occasion when used above, halo, cyano, —N₃, —NO₂, —ONO₂ or -A¹-R⁵, wherein:
A¹ represents a spacer group selected from —C(Z)A²-, —N(R⁶)A³-, —OA⁴-, —S— or —S(O)ₙA⁵-, in which:
A² represents a single bond, —O—, —S—, —N(R⁶)A⁶- or —C(Z)-;
A³ represents A⁶, —C(Z)N(R⁶)C(Z)N(R⁶)—, —C(Z)N(R⁶)C(Z)O—, —C(Z)N(R⁶)S(O)ₙN(R⁶)—, —C(Z)S—, —S(O)ₙ—, —S(O)ₙN(R⁶)C(Z)N(R⁶)—, —S(O)ₙN(R⁶)C(Z)O— or —S(O)ₙN(R⁶)S(O)ₙN(R⁶)—;
A⁴ represents A⁶ or —S(O)ₙ—;
A⁵ represents a single bond, —N(R⁶)— or —O—;
A⁶ represents, on each occasion when used above, a single bond, —C(Z)-, —C(Z)O—, —C(Z)N(R⁶)—, —S(O)ₙN(R⁶)— or —S(O)ₙO—; and
Z represents, on each occasion when used above, a substituent connected by a double bond, which is selected from ═O, ═S, ═NR⁵, ═NN(R⁵)(R⁶), ═NOR⁵, ═NS(O)₂N(R⁵)(R⁶), —NCN, ═C(H)NO₂ and ═C(R⁵)(R⁶);
R⁵ and R⁶ independently represent, on each occasion when used above,
(a) hydrogen;
(b) C₁₋₈ alkyl, optionally substituted by one or more substituents selected from X², Q, C₁₋₈ alkyl (optionally substituted by one or more substituents selected from X², C₁₋₈ alkyl, an aryl group, a heterocyclic group and Q), an aryl group and a heterocyclic group (which latter two groups are optionally substituted by one or more substituents selected from X², C₁₋₈ alkyl, an aryl group and a heterocyclic group); or
(c) an aryl group or a heterocyclic group, both of which are optionally substituted by one or more substituents selected from X², C₁₋₈ alkyl (optionally substituted by one or more substituents selected from X², C₁₋₈ alkyl, an aryl group, a heterocyclic group and Q), an aryl group and a heterocyclic group (which latter two groups are optionally substituted by one or more substituents selected from X², C₁₋₈ alkyl, an aryl group and a heterocyclic group); or
   R⁵ and R⁶ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X², C₁₋₈ alkyl, an aryl group, a heterocyclic group (which latter three groups are optionally substituted as described in (b) and (c) above, respectively) and, provided that the ring that R⁵ and R⁶ may together be part of is not aromatic in character, Q;
   X² represents, on each occasion when used above, halo, cyano, —N₃, —NO₂, —ONO₂ or -A⁷-R⁷, wherein:
   A⁷ represents a spacer group selected from —C(Q)A⁸-, —N(R⁸)A⁹-, —OA¹⁰-, —S— or —S(O)ₙA¹¹-, in which:
   A⁸ represents a single bond, —O—, —S—, —N(R⁸)— or —C(Q)-;
   A⁹ represents A¹², —C(Q)N(R⁸)C(Q)N(R⁸)—, —C(Q)N(R⁸)C(Q)O—, —C(Q)N(R⁸)S(O)ₙN(R⁸)—, —C(Q)S—, —S(O)ₙ—, —S(O)ₙN(R⁸)C(Q)N(R⁸)—, —S(O)ₙN(R⁸)C(Q)O— or —S(O)ₙN(R⁸)S(O)ₙN(R⁸)—;
   A¹⁰ represents A¹² or —S(O)ₙ—;
   A¹¹ represents a single bond, —N(R⁸)— or —O—;
   A¹² represents, on each occasion when used above, a single bond, —C(Q)-, —C(Q)O—, —C(Q)N(R⁸)—, —S(O)ₙN(R⁸)— or —S(O)ₙO—;
   Q represents, on each occasion when used above, a substituent connected by a double bond, which is selected from ═O, ═S, ═NR⁷, ═NN(R⁷)(R⁸), ═NOR⁷, —NS(O)₂N(R⁷)(R⁸), ═NCN, ═C(H)NO₂ and ═C(R⁷)(R⁸);
   R⁷ and R⁸ independently represent, on each occasion when used herein,
(i) hydrogen;
(ii) an aryl group or a heterocyclic group, both of which may be substituted by one or more substituents selected from X³, C₁₋₈ alkyl, an aryl group and a heterocyclic group (and which latter three groups are themselves optionally substituted by one or more substituents selected from halo, hydroxy, —R⁹, —OR⁹ and, provided that the group is not aromatic in nature, ═O); or
(iii) C₁₋₈ alkyl, optionally substituted by one or more substituents selected from X³ and W; or
   R⁷ and R⁸ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X³, C₁₋₈ alkyl, an aryl group, a heterocyclic group and, provided that the ring that R⁷ and R⁸ may together be part of is not aromatic in character, W;
   X³ represents, on each occasion when used above, halo, cyano, —N₃, —NO₂, —ONO₂ or -A¹³-R¹⁰, wherein:
   A represents a spacer group selected from —C(W)A¹⁴-, —N(R¹¹)A¹⁵-, —OA¹⁶-, —S— or —S(O)ₙA¹⁷-, in which:
   A¹⁴ represents a single bond, —O—, —S—, —N(R¹¹)— or —C(W)—;
   A¹⁵ represents A¹⁸, —C(W)N(R¹¹)C(W)N(R¹¹)—, —C(W)N(R¹¹)C(W)O—, —C(W)N(R¹¹)S(O)ₙN(R¹¹)—, —C(W)S—, —S(O)ₙ—, —S(O)ₙN(R¹¹)C(W)N(R¹¹)—, —S(O)ₙN(R¹¹)C(W)O— or —S(O)ₙN(R¹¹)S(O)ₙN(R¹¹)—;
   A¹⁶ represents A¹⁸ or —S(O)ₙ—;
   A¹⁷ represents a single bond, —N(R¹¹)— or —O—;
   A¹⁸ represents, on each occasion when used above, a single bond, —C(W)—, —C(W)O—, —C(W)N(R¹¹)—, —S(O)ₙN(R¹¹)— or —S(O)ₙO—;
   R⁹ represents, on each occasion when used above, C₁₋₆ alkyl optionally substituted by one or more fluoro atoms;
   W represents, on each occasion when used above, a substituent connected by a double bond, which is selected from ═O, ═S, ═NR¹⁰, ═NN(R¹⁰)(R¹¹), ═NOR¹⁰, ═NS(O)₂N(R¹⁰)(R¹¹), ═NCN, ═C(H)NO₂ and ═C(R¹⁰)(R¹¹);
   R¹⁰ and R¹¹ independently represent, on each occasion when used above:
(1) hydrogen;
(2) an aryl group or a heterocyclic group, both of which may be substituted by one or more substituents selected from X⁴, C₁₋₈ alkyl, methylenedioxy, difluoromethylenedioxy and dimethylmethylenedioxy; or
(3) C₁₋₈ alkyl, optionally substituted by one or more substituents selected from X⁴, ═O, ═S, ═NR¹², ═NN(R¹²)(R¹³), NOR¹², ═NS(O)₂N(R¹²)(R¹³), ═NCN, ═C(H)NO₂ and ═C(R¹²)(R¹³); or
   R¹⁰ and R¹¹ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X⁴ and, provided that the ring that R¹⁰ and R¹¹ may together be part of is not aromatic in character, ═O, ═S, ═NR¹², ═NN(R¹²)(R¹³), ═NOR¹², ═NS(O)₂N(R¹²)(R¹³), ═NCN, ═C(H)NO₂ and ═C(R¹²)(R¹³);
   X⁴ represents, on each occasion when used above, halo, cyano, —N₃, —NO₂, —ONO₂ or -A¹⁹-R¹², wherein:
   A¹⁹ represents a spacer group selected from —C(O)A²⁰-, —N(R¹³)A²¹, —OA²²-, —S— or —S(O)ₙA²³-, in which:
   A²⁰ represents a single bond, —O—, —S—, —N(R¹³)— or —C(O)—;
   A²¹ represents A²⁴, —C(O)N(R¹³)C(O)N(R¹³)—, —C(O)N(R¹³)C(O)O—, —C(O)N(R¹³)S(O)ₙN(R³)—, —C(O)S—, —S(O)ₙ—, —S(O)ₙN(R¹³)C(O)N(R¹³)—, —S(O)ₙN(R¹³)C(O)O— or —S(O)ₙN(R¹³)S(O)ₙN(R¹³)—;
   A²² represents A²⁴ or —S(O)ₙ—;
   A²³ represents a single bond, —N(R¹³)— or —O—;
   A²⁴ represents, on each occasion when used above, a single bond, —C(O)—, —C(O)O—, —C(O)N(R¹³)—, —S(O)ₙN(R¹³)— or —S(O)ₙO—;
   R¹² and R¹³ independently represent, on each occasion when used above:
   (A) hydrogen; or
   (B) C₁₋₆ alkyl, optionally substituted by one or more substituents selected from halo, —N(R¹⁴)R¹⁵, —OR¹⁵ and ═O;
   n represents, on each occasion when used above, 1 or 2;
   R³ and R⁴ independently represent H or C₁₋₆ alkyl optionally substituted by one or more substituents selected from halo, C₁₋₆ alkyl, cyano, —NO₂, —ONO₂, —N(R¹⁴)R¹⁵, —OR¹⁵, ═O, aryl and heteroaryl; and
   R¹⁴ and R¹⁵ independently represent, on each occasion when used above, H or C₁₋₄ alkyl,
   or a pharmaceutically acceptable salt thereof,
   for the manufacture of a medicament for the treatment of a disease in which inhibition of the activity of a lipoxygenase, and particularly 15-lipoxygenase, is desired and/or required.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of formula I contain double bonds and may thus exist as E (entgegen) and Z (zusammen) geometric isomers about each individual double bond. All such isomers and mixtures thereof are included within the scope of the invention.

Compounds of formula I may also exhibit tautomerism. All tautomeric forms and mixtures thereof are included within the scope of the invention, whether such tautomerism would lead to a loss or gain of aromaticity or otherwise.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q} cycloalkyl group). C_{3-q} cycloalkyl groups that may be mentioned include monocyclic or bicyclic alkyl groups, which cycloalkyl groups may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{3-q} cycloalkenyl, a C₈ cycloalkynyl or, more particularly, a C_{2-q} alkenyl or a C_{2-q} alkynyl group). Further in the case where the substituent is another cyclic compound, then the cyclic substituent may be attached through a single atom on the cycloalkyl group, forming a so-called “spiro”-compound.

The term “halo”, when used herein, includes fluoro, chloro, bromo and iodo.

For the avoidance of doubt, in cases in which the identity of two or more substituents in a compound of formula I may be the same, the actual identities of the respective substituents are not in any way interdependent. For example, in the situation in which R¹ and R² are both aryl groups substituted by one or more C₁₋₈ alkyl groups, the alkyl groups in question may be the same or different. Similarly, when groups are substituted by more than one substituent as defined herein, the identities of those individual substituents are not to be regarded as being interdependent. For example, when R¹ and/or R² represents e.g. an aryl group substituted by X¹ in addition to, for example, a C₁₋₈ alkyl group, which latter group is substituted by X¹, the identities of the two X¹ groups are not to be regarded as being interdependent.

Aryl groups that may be mentioned include C₆₋₁₃ (e.g. C₆₋₁₀ aryl) groups. Such groups may be monocyclic or bicyclic and have between 6 and 13 (e.g. 10) ring carbon atoms, in which at least one ring is aromatic. C₆₋₁₃ aryl groups include phenyl, naphthyl and the like, such as fluorenyl and, more particularly, 1,2,3,4-tetrahydronaphthyl, indanyl and indenyl. The point of attachment of aryl groups may be via any atom of the ring system. However, when aryl groups are bicyclic or tricyclic, they are preferably linked to the rest of the molecule via an aromatic ring. Preferred aryl groups include phenyl groups.

Heterocyclic groups that may be mentioned include those in which at least one (e.g. one to four) of the atoms in the ring system is other than carbon (i.e. a heteroatom), and in which the total number of atoms in the ring system is between three and twelve (e.g. five and ten). Heterocyclic groups may be fully saturated, wholly aromatic, partly aromatic and/or mono-, bi- or tricyclic in character, though, in the latter case, preferably at least one of the rings is aromatic. Further, non-aromatic heterocyclic groups may be unsaturated (containing one or more double and/or triple bonds) and/or bridged. Heterocyclic groups that may be mentioned include acridinyl, aziridinyl, azetidinyl, benzimidazolyl, benzodioxanyl, benzodioxepinyl, benzodioxolyl (including 1,3-benzodioxolyl), benzofuranyl, benzofurazanyl, benzothiazolyl (including 2,1,3-benzothiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, dihydropyranyl, dihydropyridyl, dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), furanyl, hydantoinyl, imidazolidinyl, imidazolinyl, imidazolyl, imidazo[1,2-a]pyridyl, indazolyl, indolinyl, indolyl, isobenzofuranyl, isochromanyl, isoindolinyl, isoindolyl, isoquinolinyl, isothiazolyl, isoxazolyl, maleimidolyl, morpholinyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, oxetanyl, oxindolyl, oxiranyl, phenazinyl, phenothiazinyl, phthalazinyl, piperazinyl, piperidinyl, pteridinyl, purinyl, pyranyl, pyrazinyl, pyrazolidinyl, pyrazolyl, pyridazinyl, pyridyl, pyrimidinyl, pyrrolidinonyl, pyrrolidinyl, pyrrolinyl, pyrrolyl, quinazolinyl, quinolinyl, quinolizinyl, quinoxalinyl, quinuclidinyl, 3-sulfolenyl, tetrahydropyranyl, tetrahydrofuranyl, tetrahydroisoquinolinyl (including 1,2,3,4-tetrahydroisoquinolinyl and 5,6,7,8-tetrahydroisoquinolinyl), tetrahydropyridyl, tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thietanyl, thiiranyl, thiolanyl, thiochromanyl, thiomorpholinyl, thienyl, triazolyl (including 1,2,3-triazolyl, 1,2,4-triazolyl and 1,3,4-triazolyl), trithianyl (including 1,3,5-trithianyl), tropanyl and the like. Other heterocyclic groups that may be mentioned include 7-azabicyclo[2.2.1]heptanyl, 6-azabicyclo[3.1.1]heptanyl, 6-azabicyclo-[3.2.1]octanyl, 8-azabicyclo[3.2.1]octanyl, dihydropyrrolyl (including 2,5-dihydropyrrolyl), 7-oxabicyclo[2.2.1]heptanyl, 6-oxabicyclo[3.2.1]-octanyl and sulfolanyl.

For the avoidance of doubt, the term “bicyclic”, when employed in the context of cycloalkyl and heterocyclic groups refers to such groups in which the second ring is formed between two adjacent atoms of the first ring. The term “bridged”, when employed in the context of cycloalkyl or non-aromatic heterocyclic groups refers to monocyclic or bicyclic groups in which two non-adjacent atoms are linked by either an alkylene or heteroalkylene chain (as appropriate).

Heteroatoms that may be mentioned include phosphorus, silicon, boron, tellurium, preferably selenium and, more preferably, oxygen, nitrogen and/or sulfur.

Substituents on heterocyclic groups may, where appropriate, be located on any atom in the ring system including a heteroatom. The point of attachment of heterocyclic groups may be via any atom in the ring system including (where appropriate) a heteroatom, or an atom on any fused carbocyclic ring that may be present as part of the ring system. Heterocyclic groups may also be in the N- or S-oxidised form.

Heteroaryl groups that R¹ and R² may represent include any of the ring systems mentioned above that are either wholly or partly aromatic in their character. This is provided that, in the latter case, the point of attachment of the heteroaryl group to the rest of the molecule is via an atom in the aromatic part of the ring system.

Compounds of the invention that may be mentioned include those in which R³ and R⁴ independently represent H or C₁₋₆ alkyl optionally substituted by one or more substituents selected from halo, C₁₋₆ alkyl, cyano, —NO₂, —ONO₂, —N(R¹⁴)R¹⁵, —OR¹⁵ and ═O.

Further compounds of the invention that may be mentioned include those in which:

A² represents a single bond, —O—, —S— or —N(R⁶)A⁶-;
A⁸ represents a single bond, —O—, —S— or —N(R⁸)—;
A¹⁴ represents a single bond, —O—, —S— or —N(R¹¹)—; and/or
A²⁰ represents a single bond, —O—, —S— or —N(R¹³)—.

Preferred compounds of formula I include those in which:

when A¹ represents —C(Z)A²-, A² represents a single bond, —O—, —S— or —N(R⁶)—;
when A¹ represents —N(R⁶)A³-, A³ represents A⁶, —C(Z)S— or —S(O)ₙ—;
when R¹ and/or R² are substituted by an alkyl group, an aryl group or a heterocyclic group, which latter three groups are substituted by one or more alkyl, aryl or heterocyclic groups, and which latter three groups are themselves substituted by an alkyl group, then that alkyl group is not cyclic in character;
when A⁷ represents —N(R⁸)A⁹-, A⁹ represents A¹², —C(Q)S— or —S(O)ₙ—;
when R⁷ and/or R⁸ represent an optionally substituted aryl group or an optionally substituted heterocyclic group, and the optional substituent is X³, then X³ represents halo, cyano or —NO₂;
when R⁷ and/or R⁸ represent a C₁₋₈ alkyl group, then that group is optionally substituted by one or more substituents selected from halo, —N(R¹⁶)R¹⁷, —OR¹⁷ and ═O, in which R¹⁶ and R¹⁷ independently represent H or C₁₋₄ alkyl.

When R⁷ and/or R⁸ represent a C₁₋₈ alkyl group, then that group is preferably optionally substituted by one or more substituents selected from halo, —NH₂, —N(H)Me, —N(H)Et, —N(H)iPr, —NMe₂, —N(Me)Et, —N(Me)iPr, —NEt₂, —OH, —OMe, —OEt, —OiPr and ═O.

Preferred alkyl groups that R⁷ and R⁸ may represent include C₁₋₆ (such as C₁₋₄) alkyl.

More preferred compounds of formula I include those in which R¹ and/or R² represent an optionally substituted pyrrolidinyl, piperidinyl, oxindolyl and, more preferably, optionally substituted phenyl, naphthyl, pyrrolyl, furanyl, thienyl (e.g. thien-2-yl or thien-3-yl), pyrazolyl, imidazolyl (e.g 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazin-yl, pyrimidinyl, pyrazinyl, indazolyl, benzimidazolyl, quinazolinyl, quinoxalinyl, 1,3-benzodioxolyl and/or benzodioxanyl group. Other compounds of formula I that may be mentioned include those in which R¹ and/or R² represents optionally substituted indazolyl, tetrazolyl and/or benzothiazolyl. Particularly preferred R¹ and/or R² groups include optionally substituted phenyl, thienyl, pyrazolyl, pyrazinyl, pyridyl, 1,3-benzodioxolyl and/or quinoxalinyl.

Such groups are optionally substituted by one or more substituents selected from:

halo (e.g. fluoro, chloro or bromo);

### —NO₂;

cyano;
C₁₋₆ alkyl, which alkyl group may be linear or branched (e.g. C₁₋₄ alkyl (including methyl, ethyl, n-propyl, i-propyl, n-butyl or t-butyl), n-pentyl, i-pentyl, n-hexyl or i-hexyl), cyclic (e.g. cyclopropyl, cyclobutyl, cyclopentyl or cyclohexyl), part-cyclic (e.g. cyclopropylmethyl), unsaturated (e.g. 1-propenyl, 2-propenyl, 1-butenyl, 2-butenyl, 3-butenyl, 1-pentenyl, 2-pentenyl, 4-pentenyl or 5-hexenyl) and/or optionally substituted with one or more halo (e.g. fluoro) group (so forming, for example, fluoromethyl, difluoromethyl or trifluoromethyl);
phenyl;
a heterocyclic group selected from a pyrrolidinyl (including 1-pyrrolidinyl, 2-pyrrolidinyl and 3-pyrrolidinyl), a piperidinyl (including 1-piperidinyl, 2-piperidinyl, 3-piperidinyl, 4-piperidinyl and 1-methyl-4-piperidinyl), a piperazinyl (including 1-piperazinyl and 4-methyl-1-piperazinyl), a tetrahydrofuranyl (including 2-tetrahydrofuranyl and 3-tetrahydrofuranyl), a tetrahydropyranyl (including 1-tetrahydropyranyl, 2-tetrahydropyranyl and 3-tetrahydropyranyl), or a morpholinyl (e.g. 4-morpholinyl), group;

### —OR¹⁸;

### —N(R¹⁸)R¹⁹;

### —C(O)R¹⁸;

### —C(O)OR¹⁸;

### —C(O)N(R¹⁸)R¹⁹;

### —S(O)ₘR²⁰;

—S(O)₂N(R¹⁸)R¹⁹; and/or

### —N(R¹⁸)S(O)₂R²⁰,

wherein R¹⁸ and R¹⁹ independently represent, on each occasion when used above, H, phenyl or C₁₋₆ alkyl, such as methyl, ethyl, n-propyl, i-propyl, n-butyl or t-butyl;
R²⁰ represents, on each occasion when used above, C₁₋₄ alkyl, such as methyl; and
m represents 0, 1 or 2.
R¹ represents an aryl (e.g. phenyl) group or a heteroaryl group, both of which are optionally substituted by one or more (e.g. one to three) substituents selected from X¹, aryl (e.g. phenyl) and C₁₋₆ (e.g. C₁₋₄) alkyl (e.g. methyl or t-butyl), which alkyl group is optionally substituted by one or more groups selected from X¹;
R² represents an aryl (e.g. phenyl) group or a heteroaryl group, both of which are optionally substituted by one or more (e.g. one or two) substituents selected from X¹, a heterocyclic group (such as a morpholinyl group (e.g. 4-morpholinyl)) and C₁₋₃ alkyl (e.g. methyl or ethyl), which alkyl group is optionally substituted by X¹;
X¹ represents halo (e.g. bromo, chloro or fluoro), —NO₂, -A¹-R¹ or cyano;
A¹ represents —C(Z)A²- or, more preferably, —N(R⁶)A³-, —OA⁴- or S(O)ₙA⁵-;
A² represents a single bond, —O— or —N(R⁶)A⁶-;
A³ represents A⁶ or —S(O)ₙ—;
A⁴ represents A⁶;
A⁵ represents a single bond or, more preferably, —N(R⁶)—;
A⁶ represents a single bond;
n represents 2;
R⁵ represents H, aryl (e.g. phenyl) or C₁₋₃ alkyl (e.g. methyl), which alkyl group is substituted by one or more groups selected from X² or, more preferably, unsubstituted;
R⁶ represents H or C₁₋₃ alkyl (e.g. methyl);
X² represents halo (e.g. fluoro);
R³ represents H or C₁₋₃ alkyl (e.g. methyl);
R⁴ represents H or C₁₋₃ alkyl (such as methyl, ethyl, propyl, butyl (e.g. isobutyl), pentyl (e.g. isopentyl)), which alkyl group is optionally substituted by one or more (e.g. one) substituents selected from aryl (e.g. phenyl) and —OR¹⁵;
R¹⁵ represents H or, more preferably, C₁₋₂ alkyl (e.g. methyl);
Z, Q and W independently represent ═O.

When R¹ represents a heterocyclic group, it is preferably a pyrazinyl group, such as a 2-pyrazinyl group (e.g. 5-methylpyrazin-2-yl) or, more preferably, a thienyl group, such as a 2-thienyl group, a pyrazolyl group, such as a 4-pyrazolyl or, more particularly, a 3-pyrazolyl group (e.g. (1,3,5-trimethyl)pyrazol-4-yl or, more particularly, 5-methylpyrazol-3-yl) or a pyridyl group, such as a 3-pyridyl or, more particularly, a 2-pyridyl group (e.g. 5-bromopyrid-3-yl, 4-methylpyrid-3-yl, 6-chloropyrid-2-yl, 5-methylpyrid-3-yl, 6-hydroxypyrid-3-yl, 6-methylpyrid-2-yl, 2-methylpyrid-3-yl, 3-methylpyrid-2-yl, 6-chloropyrid-3-yl or 6-methylpyrid-3-yl). When R² represents a heterocyclic group, it is preferably a quinoxalinyl group, such as a 2-quinoxalinyl group, a 1,3-benzodioxolyl group (e.g. 1,3-benzodioxol-5-yl) or, more preferably, a pyridyl group, such as a 4-pyridyl or, more particularly, a 2-pyridyl group.

Preferred optional substituents on R¹ and R² include —SO₂N(H)CH₃, —N(CH₃)₂, morpholinyl (e.g. 4-morpholinyl) and, more preferably, halo (e.g. fluoro, chloro and bromo), cyano, hydroxyl, amino, —NO₂, C₁₋₄ alkyl (particularly ethyl and, more particularly, methyl and t-butyl), C₁₋₄ alkoxy (particularly methoxy), phenyl, phenoxy, trifluoromethyl, —N(H)SO₂CH₃, —SO₂NH₂ and —SO₂N(CH₃)₂.

Particularly preferred values of R¹ include thienyl; pyrazolyl (such as 4-pyrazolyl or, more particularly, 3-pyrazolyl), which pyrazolyl group is substituted by one or more methyl groups or is, more preferably, unsubstituted; pyridyl (such as 3-pyridyl or, more preferably, 2-pyridyl), which pyridyl group is substituted by one or more (e.g. one) substituents selected from bromo, chloro, methyl and hydroxyl or is, more preferably, unsubstituted; and phenyl, which phenyl group is optionally substituted by one or more (e.g. one to three) substituents selected from —SO₂N(H)CH₃, —N(CH₃)₂ and, more preferably, methyl, t-butyl, methoxy, fluoro, chloro, bromo, trifluoromethyl, phenyl, hydroxyl, amino, —NO₂, —SO₂NH₂ and —SO₂N(CH₃)₂.

Particularly preferred values of R² include 4-pyridyl, 2-quinoxalinyl and, more particularly, 2-pyridyl and phenyl, which phenyl group is optionally substituted by one or more substituents selected from cyano, morpholinyl (e.g. 4-morpholinyl), —N(CH₃)₂, ethyl and, more preferably, methyl, phenoxy, —N(H)SO₂CH₃, methoxy, fluoro, chloro, bromo, trifluoromethyl, hydroxyl, —NO₂, —SO₂NH₂ and —SO₂N(CH₃)₂.

Substituents on phenyl groups that R¹ may represent may be located at any position on the phenyl ring and preferably in the 2-, 3-, 4- and/or 5-position relative to the point of attachment of that phenyl group to the rest of the molecule.

Substituents on phenyl groups that R² may represent may be located at any position on the phenyl ring and preferably in the 3- and/or 4-position relative to the point of attachment of that phenyl group to the rest of the molecule.

Substituents on phenyl groups that R² and, particularly, R¹ may represent are preferably located at the 3-position(s) relative to the point of attachment of that group to the rest of the molecule.

When R³ and R⁴ represent C₁₋₆ alkyl, preferred substituents on such alkyl groups include phenyl and, more preferably, halo, C₁₋₆ alkyl (e.g. C₁₋₃ alkyl), cyano, —NO₂, —ONO₂, —NH₂, —N(H)Me, —N(H)Et, —N(H)iPr, —NMe₂, —N(Me)Et, —N(Me)iPr, —NEt₂, —OH, —OMe, —OEt, —OiPr and ═O. More preferred substituents include phenyl, methoxy and, more particularly, cyano and —NO₂.

Preferred alkyl groups that R³ and R⁴ may represent include C₁₋₃ alkyl groups. For example, R³ and/or R⁴ may represent a methyl group.

Preferred values of R⁴ include H, methyl, ethyl, isobutyl, isopentyl, benzyl and methoxyethyl.

More preferred values of R³ and R⁴ include methyl and, particularly, H.

Particularly preferred compounds of formula I include those of the examples described hereinafter.

Compounds of formula I may be made in accordance with techniques that are well known to those skilled in the art, for example as described hereinafter.

According to a further aspect of the invention there is provided a process for the preparation of a compound of formula I, which process comprises:

(i) reaction of a compound of formula II,

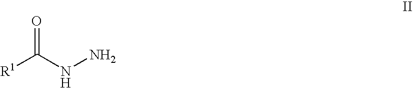

wherein R¹ is as hereinbefore defined, or an acid addition (e.g. HCl) salt thereof, with a compound of formula III,

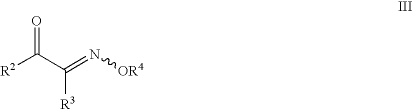

wherein the squiggly bond, R², R³ and R⁴ are as hereinbefore defined. Such a reaction may, for example, be carried out:
(a) in the case of a free base of formula II, by heating, optionally in the presence of a catalytic amount of acid (e.g. an organic acid, such as acetic acid, or an inorganic acid, such as sulfuric acid), and an appropriate organic solvent (e.g. a lower alkyl alcohol, such as methanol or ethanol), for example as described in Misra et al, J. Indian Chem. Soc., 1962, 39, 763-764, Giammanco et al, Annali di Chimica (Rome), 1961, 51, 777-784 and ibid., 1961, 51, 175-179; and
(b) in the case of an acid addition salt, at between room temperature and around 50° C. in the presence of a suitable base (e.g. sodium or potassium acetate) and an appropriate solvent system (such as ethanol or water), for example as described in Rupe et al, Chem. Ber., 1909, 42, 4715-4720 and Lalezari, J. Org. Chem., 1968, 33, 4281-4283;
   (ii) reaction of a compound of formula IV,

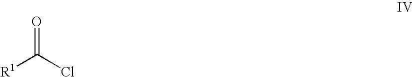

wherein R¹ is as hereinbefore defined with a compound of formula V,

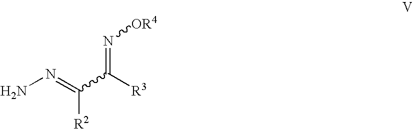

wherein the squiggly bonds, R², R³ and R⁴ are as hereinbefore defined, for example as described in Dey, J. Chem. Soc., 1914, 105, 1039-1046, Forster, ibid., 1912, 101, 2234-2240 and Neunhoeffer, Liebeigs Ann. Chem. 1976, 153-162;
(iii) reaction of a compound of formula VI,

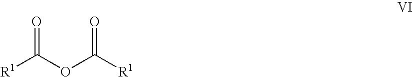

wherein R¹ is as hereinbefore defined with a compound of formula V as hereinbefore defined, for example as described in Neunhoeffer, Liebeigs Ann. Chem. 1976, 153-162;
(iv) ring opening of a compound of formula VII,

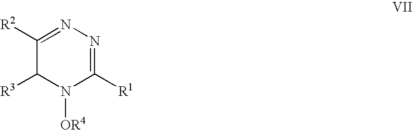

wherein R¹, R², R³ and R⁴ are as hereinbefore defined, for example at around room temperature in the presence of a suitable base (e.g. sodium hydroxide) and an appropriate solvent (e.g. water), as described in Neunhoeffer, Liebeigs Ann. Chem. 1976, 153-162;
(v) reaction of a compound of formula VIII,

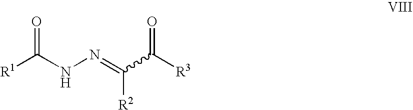

wherein the squiggly bond, R¹, R² and R³ are as hereinbefore defined with a compound of formula IX,

R⁴ONH₂ IX

wherein R⁴ is as hereinbefore defined, or an acid addition (e.g. HCl) salt thereof, for example at between around 0° C. and room temperature in the presence of a suitable base (e.g. KOH) and an appropriate solvent system (e.g. ethanol/water), for example as described in Metze, Chem. Ber. 1958, 1861-1866;
(vi) for compounds of formula I in which R⁴ represents optionally substituted C₁₋₆ alkyl, reaction of a corresponding compound of formula I in which R⁴ represents H with a compound of formula X,

R^{4a}L¹ X

wherein L¹ is a suitable leaving group (e.g. halo, such as iodo) and R^{4a} is C₁₋₆ alkyl optionally substituted by one or more substituents selected from halo, C₁₋₆ alkyl, cyano, —NO₂, —ONO₂, —N(R¹⁴)R¹⁵, —OR¹⁵, ═O, aryl and heteroaryl, for example, in the case when L¹ is iodo, at low temperature, such as around 0° C. in the presence of a suitable catalyst (e.g. silver (I) oxide) and an appropriate solvent (e.g. methanol and/or dichloromethane), for example as described in Buehler, J. Org. Chem., 1967, 32, 261-265 and Adams et al, J. Chem. Soc., Perkin Trans. 2, 1991, 1809-1818. Alternatively, the reaction may be performed in the presence of a suitable base (e.g. KOH, NaOH, K₂CO₃ and/or sodium ethoxide), in the presence of a suitable solvent system (e.g. toluene, DMF, DMSO, EtOH and/or water). In the event that biphasic reaction conditions are employed, a phase transfer catalyst (e.g. tetrabutylammonium bromide) may be employed. Preferred combinations of base and solvent include EtOH and sodium ethoxide, KOH and DMSO, NaOH and toluene/water and K₂CO₃ and DMF; or
(vii) reaction of a compound of formula XI,

R¹L² XI

wherein L² is a suitable leaving group (e.g. halo) and R¹ is as hereinbefore defined with a compound of formula V as hereinbefore defined in the presence of carbon monoxide (or another suitable CO source such as Mo(CO)₆ or CO₂(CO)₈) for example by heating in the presence of an appropriate metal catalysts (e.g. Pd) and an appropriate solvent (e.g. DMF).

Compounds of formulae II, III, IV, V, VI, VII, VIII, IX, X and XI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to infer alia “Comprehensive Organic Synthesis” by B. M. Trost and I. Fleming, Pergamon Press, 1991.

For example, compounds of formula III may be prepared by a variety of techniques, for example as described hereinafter.

Further, the substituents R¹, R², R³ and R⁴ as hereinbefore defined may be modified one or more times, after or during the processes described above for preparation of compounds of formula I by way of methods that are well known to those skilled in the art. Examples of such methods include substitutions, reductions, oxidations, alkylations, hydrolyses, esterifications, and etherifications. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

Compounds of the formula I may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Compounds of the formula I and salts thereof are useful because they possess pharmacological activity. Such compounds/salts are therefore indicated as pharmaceuticals.

Certain compounds of formula I have not been disclosed before for use as pharmaceuticals. According to a further aspect of the invention there is provided a compound of formula I as hereinbefore defined, or a pharmaceutically-acceptable salt thereof, for use as a pharmaceutical, provided that, when R⁴ represents H and:

(A) R³ represents H and:
(I) R² represents phenyl, then R¹ does not represent 2-furanyl, 4-pyridyl, 3-(5-methylisooxazolyl), phenyl, or 3-nitro-, 2-hydroxy-, 2-hydroxy-3-methyl-, 4-(thienyl)-, 2-hydroxy-5-methyl- or 4-hydroxyphenyl;
(II) R² represents 4-chlorophenyl, then R¹ does not represent 2-furanyl, 4-pyridyl, phenyl, or 2-hydroxy-5-methyl-, 4-hydroxy-, 2-hydroxy-3-methyl- or 2-hydroxyphenyl;
(III) R² represents 4-methylphenyl, then R¹ does not represent 4-pyridyl, phenyl, or 3-nitro-, 2-hydroxy-5-methyl-, 4-hydroxy-, 2-hydroxy- or 4-(thienyl)phenyl; or
(IV) R² represents 2-furanyl or 2-benzofuranyl, then R¹ does not represent 4-pyridyl or 3-(5-methylisooxazolyl); and
   (B) R³ represents methyl and:
(1) R² represents phenyl, then R¹ does not represent N-(4-bromophenyl)-2-amino-, N-(2-methoxyphenyl)-2-amino-, N-(2-ethoxyphenyl)-2-amino-, N-(3-chlorophenyl)-2-amino-, N-(4-methylphenyl)-2-amino-, N-(3-methylphenyl)-2-amino-, N-(2-methylphenyl)-2-amino- or N-(phenyl)-2-aminophenyl; or
(2) R¹ represents 4-chlorophenyl, then R¹ does not represent 4-pyridyl, phenyl, or 3-nitro-, 2-hydroxy-5-methyl-, 4-hydroxy-, 2-hydroxy- or 2-hydroxy-3-methylphenyl.

Certain compounds of formula I are novel per se. According to a further aspect of the invention there is provided a compound of formula I as defined above, or a pharmaceutically-acceptable salt thereof, with the additional provisos that, when R⁴ represents H, R² represents phenyl and:

(a) R³ represents H, then R¹ does not represent 2-pyridyl, or 3-bromo-, 3,4-dimethoxy- or 5-bromo-2-hydroxyphenyl; and
(b) R³ represents methyl, then R¹ does not represent 4-methoxyphenyl.

Although compounds of formula I and salts thereof may possess pharmacological activity as such, certain pharmaceutically-acceptable (e.g. “protected”) derivatives of compounds of formula I may exist or be prepared which may not possess such activity, but may be administered parenterally or orally and thereafter be metabolised in the body to form compounds of formula I. Such compounds (which may possess some pharmacological activity, provided that such activity is appreciably lower than that of the “active” compounds to which they are metabolised), may therefore be described as “prodrugs” of compounds of formula I. All prodrugs of compounds of formula I are included within the scope of the invention.

By “prodrug of a compound of formula I”, we include compounds that form a compound of formula I, in an experimentally-detectable amount, within a predetermined time (e.g. about 1 hour), following oral or parenteral administration.

Compounds of formula I and salts thereof are useful because, in particular, they may inhibit the activity of lipoxygenases, particularly 15-lipoxygenase, for example as may be demonstrated in the test described below. Compounds of formula I may thus be useful in the treatment of those conditions in which inhibition of a lipoxygenase, and particularly 15-lipoxygenase, is required.

Compounds of formula I, and pharmaceutically acceptable salts thereof, are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Accordingly, compounds of formula I may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of formula I and pharmaceutically acceptable salts thereof may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of formula I are indicated both in the therapeutic and/or prophylactic treatment of the above-mentioned conditions.

According to a further aspect of the present invention, there is provided a method of treatment of a disease in which inhibition of the activity of a lipoxygenase, and particularly 15-lipoxygenase, is desired and/or required (e.g. inflammation), which method comprises administration of a therapeutically effective amount of a compound of formula I, or a pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of formula I will normally be administered sublingually or preferably, orally, intravenously, subcutaneously, buccally, rectally, dermally, nasally, tracheally, bronchially, by any other parenteral route or via inhalation, in a pharmaceutically acceptable dosage form.

Compounds of formula I may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I as specified herein, or a pharmaceutically-acceptable salt thereof, in admixture with a pharmaceutically acceptable adjuvant, diluent or carrier.

Compounds of formula I may also be combined with other therapeutic agents that are useful in the treatment of inflammation as defined herein (e.g. NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of formula I or a pharmaceutically-acceptable salt thereof, and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of formula I or salt thereof in conjunction with the other therapeutic agent, and may thus be presented either as separate formulations, wherein at least one of those formulations comprises compound of formula I/salt, and at least one comprises the other therapeutic agent, or may be presented (i.e. formulated) as a combined preparation (i.e. presented as a single formulation including compound of formula I/salt and the other therapeutic agent).

Thus, there is further provided:

(1) a pharmaceutical formulation including a compound of formula I or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I or a pharmaceutically-acceptable salt thereof in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of formula I and salts thereof may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of formula I and salts thereof may have the advantage that they are effective and/or selective inhibitors of lipoxygenases, and particularly 15-lipoxygenase.

Compounds of formula I and salts thereof may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;
d) 5 μl of 0.125 mM arachidonic acid in PBS. The plate is then incubated for 10 minutes at room temperature;
e) the enzymatic reaction is terminated by the addition of 100 μl methanol; and
f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:

DMF dimethylformamide
DMSO dimethylsulfoxide
MS mass spectrum
NMR nuclear magnetic resonance
RT room temperature

Starting materials and chemical reagents specified in the syntheses described below are commercially available from, e.g. Sigma-Aldrich Fine Chemicals.

### General Procedures

Many of the examples below were prepared in accordance with the following general procedures.

### General Procedure X

The relevant hydrazide of formula II as described herein (2.4 mmol) was dissolved in MeOH (10 mL) and cooled to 0° C. Two drops of concentrated sulfuric acid were added followed by dropwise addition of the relevant oxime of formula III as described herein (2 mmol) dissolved in MeOH (10 mL). The reaction was heated at reflux for 16 h. The product could be isolated by chromatography or, if a precipitate is formed during the reaction, this could be filtered off, washed with MeOH and recrystallised from MeOH/water.

### General Procedure Y

The relevant hydrazide of formula II as described herein (2.4 mmol) was dissolved in EtOH (10 mL) and to it was added 2 drops of concentrated sulfuric acid, and the appropriate oxime of formula III as described herein (2 mmol). The reaction was heated at 50° C. overnight, filtered, washed with EtOH and recrystallised from EtOH/water.

### General Procedure Z

The relevant compound of formula I in which R⁴ represents H (i.e. an oxime) (1 eq.) was dissolved in EtOH (ca. 5 mL/eq.), after which sodium ethoxide (2.5M, 1.1 eq.) was added. The solution was stirred at 80° C. for 1.5 h. The solvent was removed in vacuo. The residue was suspended in DMF and the bromoalkane of formula X (1.8 eq.) was added. The mixture was stirred at RT overnight. EtOAc was added and the organic phase was washed with 3 portions of NaOH (aq., 2M) and 2 portions of CaCl₂ (aq., sat.), water and NaCl (aq., sat.). The organic phase was dried (Na₂SO₄) and the solvent removed in vacuo. The product was isolated by precipitation by first dissolving in EtOH and then adding water.

Starting materials were commercially available, but in the event that they were not, the following general procedures were employed.

### Procedure A

Isoamyl nitrite (1.83 g, 15.6 mmol) was dissolved in ice-cold ethanolic sodium ethoxide (0.9 M, 18.3 mL, 16.5 mmol). The relevant acetophenone (14.9 mmol) was added dropwise to the cooled solution and the reaction was allowed to reach room temperature and stirring was continued for 16 h. The solid formed during the course of the reaction was filtered off, washed with diethyl ether, dissolved in water and the aqueous solution acidified with glacial acetic acid. The resultant crystalline solid was filtered off and recrystallised from EtOH/water.

### Procedure B

Isoamyl nitrite (1.83 g, 15.6 mmol) was dissolved in ice-cold ethanolic sodium ethoxide (0.9 M, 18.3 mL, 16.5 mmol). The relevant acetophenone (14.9 mmol) was added dropwise to the cooled solution. The reaction was allowed to reach room temperature and stirring was continued for 16 h. Water (100 mL) was then added, and the mixture was extracted with diethyl ether. The aqueous phase was acidified with glacial acetic acid and extracted with diethyl ether. The organic phase was dried (Na₂SO₄), filtered and concentrated in vacuo. Purification by column chromatography gave the desired product.

### Procedure C

Selenium dioxide (3.0 g, 27 mmol) was dissolved in a mixture of dioxane (20 mL) and water (600 μL), and the relevant acetophenone (27 mmol) was added. The mixture was heated at reflux for 16 h, filtered through Celite®, diluted with water and adjusted to pH=4 with aqueous 4 M NaOH. A solution of hydroxylamine hydrochloride (2.3 g, 33 mmol) in aqueous NaOH (4 M, 8.3 mL, 33.2 mmol) was added and the pH was adjusted to 4 with aqueous 4 M NaOH. The reaction was left for 16 h at 50° C., diluted with water/ice and extracted with diethyl ether. The combined organic phases were washed with water, dried (Na₂SO₄), filtered and concentrated in vacuo. Purification by column chromatography gave the desired product, which was recrystallised from EtOH/water.

### Preparation of Intermediates

### Preparation 1

### 2-Oxo-2-(2-(trifluoromethyl)phenyl)acetaldehyde oxime

General procedure C was employed to give the title compound as a yellow crystalline solid in 73% yield.

¹H-NMR (300 MHz, DMSO-d₆) δ 13.00 (s, 1H), 7.97 (s, 1H), 7.84-7.61 (m, 4H).

### Preparation 2

### 2-(2-Butoxyphenyl)-2-oxoacetaldehyde oxime

General procedure C was employed to give the title compound as a yellow crystalline solid in 68% yield.

¹H-NMR (300 MHz, DMSO-d₆) δ 12.44 (s, 1H), 7.95 (s, 1H), 7.48 (dt, 1H), 7.37 (dd, 1H), 7.12 (d, 1H), 7.01 (t, 1H), 4.01 (t, 2H), 1.69-1.60 (m, 2H), 1.43-1.36 (m, 2H), 0.90 (t, 3H).

### Preparation 3

### 2-(2-Fluorophenyl)-2-oxoacetaldehyde oxime

General procedure C was employed to give the title compound as a yellow crystalline solid in 44% yield.

¹H-NMR (300 MHz, DMSO-d₆) δ 12.86 (s, 1H), 7.95 (s, 1H), 7.65-7.59 (m, 2H), 7.34-7.28 (m, 2H).

### Preparation 4

### 2-Oxo-2-(4-phenoxyphenyl)acetaldehyde oxime

General procedure B was employed to give the title compound as a yellow crystalline solid in 33% yield.

¹H-NMR (300 MHz, DMSO-d₆) δ 12.61 (s, 1H), 8.06-8.03 (m, 3H), 7.47 (t, 2H), 7.26 (t, 1H), 7.14 (d, 2H), 7.06 (d, 2H).

### Preparation 5

### 2-(2-Bromophenyl)-2-oxoacetaldehyde oxime

General procedure A was employed to give the title compound as a yellow crystalline solid in 28% yield.

¹H-NMR (300 MHz, DMSO-d₆) δ 12.95 (s, 1H), 7.90 (s, 1H), 7.68 (d, 1H), 7.48-7.40 (m, 3H).

### Preparation 6

### N-(3-(2-(Hydroxyimino)acetyl)phenyl)methanesulfonamide

General procedure C was employed to give the title compound as a light brown crystalline solid in 24% yield.

¹H-NMR (300 MHz, DMSO-d₆) δ 12.76 (bs, 1H), 10.05 (bs, 1H), 8.07 (s, 1H), 7.83 (bs, 1H), 7.80-7.76 (m, 1H), 7.58-7.51 (m, 2H), 3.09 (s, 3H).

### EXAMPLES

### Example 1

### 4-tert-Butyl-N′-(2-(hydroxyimino)-1-phenylethylidene)benzohydrazide

4-tert-Butylbenzohydrazide (100 mg, 309 μmol), 2-isonitrosoacetophenone (46 mg, 309 μmol), 2 drops of glacial acetic acid and absolute ethanol (3 mL) were mixed in a closed reaction vessel flushed with nitrogen and equipped with a stir bar. The reaction mixture was heated at 85° C. with magnetic stirring for 4 h, and then kept in a freezer overnight. The solid that formed was collected and recrystallized from EtOH/water to yield 22 mg (22%) of the desired product as a white crystalline solid.

¹H-NMR (400 MHz, DMSO-d₆) δ 13.26 (bs, 1H), 12.70 (bs, 1H), 8.52 (s, 1H), 7.87-7.80 (m, 2H), 7.80-7.73 (m, 2H), 7.60-7.53 (m, 2H), 7.48-7.41 (m, 3H), 1.32 (s, 9H). ¹³C-NMR (100.5 MHz, DMSO-d₆) δ 163.4, 155.3, 145.2, 141.5, 136.3, 130.0, 129.3, 128.5, 127.4, 127.1, 125.7, 34.8, 30.9.

### Example 2

### 3-Bromo-N′-(2-(methoxyimino)-1-phenylethylidene)benzohydrazide

3-Bromo-N′-(2-(hydroxyimino)-1-phenylethylidene)benzohydrazide (10 mg, 28.9 μmol) and methyl iodide (21 mg, 144 μmol) were dissolved in methanol/dichloromethane (1:1, 5 mL) and cooled in an icebath. Silver (I) oxide (7.4 mg, 32 μmol) was added and the reaction mixture was stirred for 2 h at 0° C. Methyl iodide (21 mg, 144 μmol) was added and the reaction was stirred at room temperature for 16 h. The mixture was filtered, concentrated and purified by chromatography to yield 6.3 mg (61%) of the desired product as a white crystalline solid.

¹H-NMR (400 MHz, CD₃CN) δ 13.16 (bs, 1H), 8.41 (s, 1H), 8.08 (t, J=2 Hz, 1H), 7.90 (d, J=9 Hz, 1H), 7.82-7.70 (m, 3H), 7.53-7.40 (m, 4H), 4.16 (s, 3H).

### Example 3

### N′-(2-(Hydroxyimino)-1-phenylethylidene)-3-methoxybenzohydrazide

General procedure X was employed to give the title compound as a white crystalline solid in 32% isolated yield.

¹H-NMR (300 MHz, DMSO-d₆) δ 13.05 (s, 1H), 12.60 (s, 1H), 8.51 (s, 1H), 7.77-7.76 (m, 2H), 7.51-7.41 (m, 6H), 7.23-7.20 (m, 1H), 3.85 (s, 3H).

### Example 4

### 3-Chloro-N′-(2-(hydroxyimino)-1-phenylethylidene)benzohydrazide

General procedure X was employed to give the title compound as a colourless powder in 45% yield.

¹H-NMR (300 MHz, DMSO-d₆) δ 12.89 (s, 1H), 12.58 (s, 1H), 8.54 (s, 1H), 7.91 (s, 1H), 7.91-7.73 (m, 4H), 7.61 (t, 1H), 7.45 (bs, 3H).

### Example 5

### N′-(2-(Hydroxyimino)-1-phenylethylidene)thiophene-2-carbohydrazide

Thiophene-2-carbohydrazide (100 mg, 703 μmol), 2-isonitroso-acetophenone (126 mg, 844 μmol), 2 drops of glacial acetic acid and absolute ethanol (4 mL) were mixed in a closed reaction vessel flushed with nitrogen and equipped with a stir bar. The reaction mixture was stirred at 85° C. for 16 h and then kept in a freezer overnight. The crystalline solid formed was collected and purified by chromatography to yield 27 mg (14% isolated yield) of the desired product as a white crystalline solid.

¹H-NMR (400 MHz, DMSO-d₆) δ 12.35 (bs, 1H), 8.53 (s, 1H), 8.12-7.75 (m, 4H), 7.46 (m, 3H), 7.26 (m, 1H).

### Example 6

### 4-Chloro-N′-(2-(hydroxyimino)-1-phenylethylidene)benzohydrazide

4-Chlorobenzoic hydrazide (200.1 mg, 1.17 mmol) was dissolved in ethanol (4 mL) in a reaction vial. To this was added 2-isonitrosoacetophenone (212 mg, 1.40 mmol) and 2 drops of acetic acid. The vial was heated at 82° C. with magnetic stirring. After 4 hours, the reaction was stopped and the reaction mixture was put in the freezer. Upon cooling a precipitate formed. This was filtered off and recrystallised from ethanol to give the desired product in 41% yield (146.3 mg, 0.48 mmol).

¹H-NMR (270 MHz, dmso-d₆) δ 13.1 (s, 1H), 12.6 (s, 1H), 8.53 (s, 1H), 7.90 (d, 2H), 7.76 (s, 2H), 7.64 (d, 2H), 7.46 (s, 3H).

### Example 7

### 2-Bromo-N′-(2-(hydroxyimino)-1-(4-phenoxyphenyl)ethylidene)benzo-hydrazide

General procedure X was employed (using the intermediate from Preparation 4) to give a mixture of isomers of the title compound as a yellow powder in 23% yield.

¹H-NMR (300 MHz, DMSO-d₆) δ 12.48 (s, 1H, minor isomer), 12.34 (s, 1H), 12.21 (s, 1H, major), 8.50 (s, 1H, minor), 8.49 (s, 1H, major), 7.78-7.37 (m, 8H), 7.22-7.00 (m, 4H), 6.90 (d, 1H).

### Example 8

### N′-(1-(2-Chlorophenyl)-2-(hydroxyimino)ethylidene)benzohydrazide

General procedure X was employed to give the title compound as a yellow powder in 15% yield.

¹H-NMR (300 MHz, DMSO-d₆) δ 12.6 (s, 1H), 12.43 (s, 1H), 8.41 (s, 1H), 7.90 (d, 2H), 7.62-7.39 (m, 7H).

### Example 9

### N′-(2-(Hydroxyimino)-1-(4-methoxyphenyl ethylidene)benzohydrazide

General procedure X was employed to give the title compound as a yellow powder in 9% yield.

¹H-NMR (300 MHz, DMSO-d₆) δ 13.1 (s, 1H), 12.59 (s, 1H), 8.50 (s, 1H), 7.88 (d, 2H), 7.78-7.70 (m, 2H), 7.66-7.52 (m, 3H), 7.01 (d, 2H), 3.81 (s, 3H).

### Example 10

### N′-(2-(Hydroxyimino)-1-(3-hydroxyphenyl)ethylidene)benzohydrazide

General procedure X was employed to give the title compound as a beige crystalline solid in 16% yield.

¹H-NMR (300 MHz, DMSO-d₆) δ 13.20 (s, 1H), 12.61 (s, 1H), 9.60 (s, 1H), 8.42 (s, 1H), 7.90 (d, 2H), 7.69-7.53 (m, 3H), 7.28-7.14 (m, 3H), 6.84 (d, 1H).

### Example 11

### N′-(1-(4-Bromophenyl)-2-(hydroxyimino)ethylidene)benzohydrazide

General procedure X was employed to give the title compound as an off-white powder in 35% yield.

¹H-NMR (300 MHz, DMSO-d₆) δ 13.16 (s, 1H), 12.64 (s, 1H), 8.53 (s, 1H), 7.89 (d, 2H), 7.75-7.54 (m, 7H).

### Example 12

### N′-(2-(Hydroxyimino)-1-phenylethylidene)-3,5-bis(trifluoromethyl)benzo-hydrazide

General procedure X was employed to give a mixture of isomers of the title compound as a colourless crystalline solid in 36% yield.

¹H-NMR (300 MHz, DMSO-d₆) δ 12.58 (s, 1H, minor), 12.46 (s, 1H), 12.27 (s, 1H, major), 8.64 (s, 1H), 8.52 (s, 2H), 8.42-8.34 (m, 1H), 7.76-7.44 (m, 5H).

### Example 13

### 2-Bromo-N′-(1-(2-fluorophenyl)-2-(hydroxyimino)ethylidene)benzohydrazide

General procedure X was employed (using the intermediate from Preparation 3) to give a mixture of isomers of the title compound as an off-white powder in 18% yield.

¹H-NMR (300 MHz, DMSO-d₆) δ 12.41 (s, 1H, minor), 12.35 (s, 1H, minor), 12.32 (s, 1H, major), 12.12 (s, 1H, major), 8.59 (s, 1H), 7.80-7.17 (m, 8H).

### Example 14

### N′-(1-(3-Chlorophenyl)-2-(hydroxyimino)ethylidene)benzohydrazide

General procedure X was employed to give the title compound as a colourless crystalline solid in 36% yield.

¹H-NMR (300 MHz, DMSO-d₆) δ 13.21 (s, 1H), 12.63 (s, 1H), 8.57 (s, 1H), 7.90 (d, 2H), 8.82 (s, 1H), 7.75 (d, 1H), 7.70-7.46 (m, 5H).

### Example 15

### N′-(1-(3-Chlorophenyl)-2-(hydroxyimino)ethylidene)-2-methylbenzo-hydrazide

General procedure X was employed to give the title compound as a colourless crystalline solid in 19% yield.

¹H-NMR (300 MHz, DMSO-d₆) δ 12.43 (s, 2H), 8.53 (s, 1H), 7.77 (d, 1H), 7.55-7.28 (m, 7H), 2.42 (s, 3H).

### Example 16

### 3-Bromo-N′-(2-(hydroxyimino)-1-(4-phenoxyphenyl)ethylidene)-benzohydrazide

General procedure X was employed (using the intermediate from Preparation 4) to give the title compound as a colourless crystalline solid in 59% yield.

¹H-NMR (300 MHz, DMSO-d₆) δ 12.79 (s, 1H), 12.57 (s, 1H), 8.53 (s, 1H), 8.05 (s, 1H), 7.86-7.76 (m, 4H), 7.51 (t, 1H), 7.43 (t, 2H), 7.19 (t, 1H), 7.07 (t, 4H).

### Example 17

### 3-Bromo-N′-(2-(hydroxyimino)-1-(3-hydroxyphenyl)ethylidene)benzohydrazide

General procedure X was employed to give the title compound as a colourless powder in 4% yield.

¹H-NMR (300 MHz, DMSO-d₆) δ 12.90 (s, 1H), 12.58 (s, 1H), 9.61 (s, 1H), 8.46 (s, 1H), 8.05 (s, 1H), 7.84 (d, 2H), 7.52 (t, 1H), 7.24-7.15 (m, 3H), 6.85 (d, 1H).

### Example 18

### 3-Bromo-N′-(1-(4-bromophenyl)-2-(hydroxyimino)ethylidene)benzohydrazide

General procedure X was employed to give the title compound as a colourless crystalline solid in 46% yield.

¹H-NMR (300 MHz, DMSO-d₆) δ 12.82 (s, 1H), 12.59 (s, 1H), 8.55 (s, 1H), 8.03 (d, 1H), 7.86-7.81 (m 2H), 7.78-7.63 (m, 4H), 7.54-7.45 (m, 1H).

### Example 19

### N′-(1-(3-Chlorophenyl)-2-(hydroxyimino ethylidene)-2-fluorobenzohydrazide

General procedure X was employed to give a mixture of isomers of the title compound as a colourless crystalline solid in 24% yield.

¹H-NMR (300 MHz, DMSO-d₆) δ 12.76 (s, 1H, major isomer), 12.53 (s, 1H), 12.42 (s, 1H, minor isomer), 8.56 (s, 1H, minor isomer), 8.51 (s, 1H, major isomer), 7.87-7.37 (m, 8H).

### Example 20

### 4-Phenyl-N′-(2-(hydroxyimino)-1-(4-phenoxyphenyl)ethylidene)benzohydrazide

General procedure X was employed (using the intermediate from Preparation 4) to give the title compound as a beige powder in 470% yield.

¹H-NMR (300 MHz, DMSO-d₆) δ 13.15 (s, 1H), 12.64 (s, 1H), 8.54 (s, 1H), 7.99 (d, 2H), 7.86-7.70 (m, 6H), 7.53 (t, 2H), 7.44 (t, 3H), 7.20 (t, 1H), 7.08 (t, 4H).

### Example 21

### 3-Bromo-N′-(2-(hydroxyimino)-1-(3-methoxyphenyl)ethylidene)benzohydrazide

General procedure X was employed to give the title compound as a colourless crystalline solid in 63% yield.

¹H-NMR (300 MHz, DMSO-d₆) δ 12.94 (s, 1H), 12.60 (s, 1H), 8.53 (s, 1H), 8.05 (s, 1H), 7.84 (d, 2H), 7.52 (t, 1H), 7.37-7.28 (m, 3H), 7.03 (d, 1H), 3.80 (s, 3H).

### Example 22

### 3-Bromo-N′-(2-(hydroxyimino)-1-(4-methoxyphenyl)ethylidene)benzohydrazide

General procedure X was employed to give the title compound as a yellow crystalline solid in 78% yield.

¹H-NMR (300 MHz, DMSO-d₆) δ 12.80 (s, 1H), 12.53 (s, 1H), 8.51 (s, 1H), 8.04 (s, 1H), 7.83 (d, 2H), 7.73 (d, 2H), 7.51 (t, 1H), 7.00 (d, 2H), 3.81 (s, 3H).

### Example 23

### N′-(1-(2-Fluorophenyl)-2-(hydroxyimino)ethylidene)-4-phenylbenzohydrazide

General procedure X was employed (using the intermediate from Preparation 3) to give the title compound as a colourless powder in 58% yield.

¹H-NMR (300 MHz, DMSO-d₆) δ 12.79 (s, 1H), 12.51 (s, 1H), 8.44 (d, 1H), 8.00 (d, 2H), 7.84 (d, 2H), 7.76 (d, 2H), 7.59-7.41 (m, 5H), 7.33-7.27 (m, 2H).

### Example 24

### 4-tert-Butyl-N′-(2-(hydroxyimino)-1-(4-phenoxyphenyl)ethylidene)benzohydrazide

General procedure X was employed (using the intermediate from Preparation 4) to give the title compound as a yellow powder in 52% yield.

¹H-NMR (300 MHz, DMSO-d₆) δ 13.18 (s, 1H), 12.62 (s, 1H), 8.52 (s, 1H), 7.86-7.79 (m, 4H), 7.57 (d, 2H), 7.44 (t, 2H), 7.20 (t, 1H), 7.10-7.04 (m, 4H), 1.33 (s, 9H).

### Example 25

### N′-(2-(Hydroxyimino)-1-(3-methoxyphenyl)ethylidene)benzohydrazide

General procedure X was employed to give the title compound as a pink powder in 45% yield.

¹H-NMR (300 MHz, DMSO-d₆) δ 13.28 (s, 1H), 12.60 (s, 1H), 8.52 (s, 1H), 7.90 (d, 2H), 7.66-7.55 (m, 3H), 7.40-7.31 (m, 3H), 7.02 (d, 1H), 3.81 (s, 3H).

### Example 26

### 3-Bromo-N′-1-(4-chlorophenyl)-2-(hydroxyimino)ethylidene)benzohydrazide

General procedure X was employed to give the title compound as a colourless powder in 75% yield.

¹H-NMR (300 MHz, DMSO-d₆) δ 12.81 (s, 1H), 12.59 (s, 1H), 8.55 (s, 1H), 8.05 (s, 1H), 7.86-8.77 (m, 4H), 7.54-7.49 (m, 3H).

### Example 27

### 4-tert-Butyl-N′-1-(2-fluorophenyl)-2-(hydroxyimino)ethylidene)benzohydrazide

General procedure X was employed (using the intermediate from Preparation 3) to give the title compound as a pink powder in 29% yield.

¹H-NMR (300 MHz, DMSO-d₆) δ 12.83 (s, 1H), 12.50 (s, 1H), 8.38 (d, 1H), 7.84 (d, 2H), 7.58-7.48 (m, 4H), 7.33-7.26 (m, 2H), 1.33 (s, 9H).

### Example 28

### N′-(1-(2-Bromophenyl)-2-(hydroxyimino)ethylidene)-4-tert-butylbenzohydrazide

General procedure X was employed (using the intermediate from Preparation 5) to give the title compound as a white powder in 18% yield.

¹H-NMR (300 MHz, DMSO-d₆) δ 12.62 (s, 1H), 12.43 (s, 1H), 8.37 (s, 1H), 7.83 (d, 2H), 7.70 (d, 1H), 7.55 (d, 2H), 7.48 (d, 2H), 7.44-7.35 (m, 1H), 1.33 (s, 9H).

### Example 29

### 3-Bromo-N′-(1-(2-chlorophenyl)-2-(hydroxyimino)ethylidene)benzohydrazide

General procedure X was employed to give the title compound as a white powder in 41% yield.

1H-NMR (300 MHz, DMSO-d₆) δ 12.36 (s, 1H), 12.24 (s, 1H), 8.55 (s, 1H), 8.09 (s, 1H), 7.86-7.80 (m, 2H), 7.61-7.44 (m, 5H).

### Example 30

### 3-Chloro-N′-(1-(2-fluorophenyl)-2-(hydroxyimino)ethylidene)benzohydrazide

General procedure X was employed (using the intermediate from Preparation 3) to give the title compound as a colourless powder in 40% yield.

¹H-NMR (300 MHz, DMSO-d₆) δ12.42 (s, 2H), 8.51 (d, 1H), 7.94 (s, 1H), 7.83 (d, 1H), 7.69 (d, 1H), 7.59-7.47 (m, 3H), 7.31-7.25 (m, 2H).

### Example 31

### N′-(1-(2-Bromophenyl)-2-(hydroxyimino ethylidene)-4-phenylbenzohydrazide

General procedure X was employed (using the intermediate from Preparation 5) to give the title compound as a colourless crystalline solid in 5% yield.

¹H-NMR (300 MHz, DMSO-d₆) δ 12.58 (s, 1H), 12.44 (s, 1H), 8.00 (d, 1H), 8.00 (d, 1H), 7.85-7.67 (m, 5H), 7.54-7.36 (m, 7H).

### Example 32

### 3-Chloro-N′-(2-(hydroxyimino)-1-(4-phenoxyphenyl)ethylidene)benzohydrazide

General procedure X was employed (using the intermediate from Preparation 4) to give the title compound as a pink powder in 70% yield.

¹H-NMR (300 MHz, DMSO-d₆) δ 12.82 (s, 1H), 12.56 (s, 1H), 8.53 (s, 1H), 7.91 (s, 1H), 7.82-7.69 (m, 4H), 7.58 (t, 1H), 7.44 (t, 2H), 7.20 (t, 1H), 7.07 (t, 4H).

### Example 33

### N′-(2-(Hydroxyimino)-1-phenylethylidene)-3,4,5-trimethoxybenzohydrazide

General procedure X was employed to give the title compound as a white powder in 76% yield.

¹H-NMR (300 MHz, DMSO-d₆) δ 12.8 (s, 1H), 12.51 (s, 1H), 8.51 (s, 1H), 7.78-7.75 (m, 2H), 7.47-7.45 (m, 3H), 7.13 (s, 2H), 3.89 (s, 6H), 3.75 (s, 3H).

### Example 34

### 2-Bromo-N′-(2-(hydroxyimino)-1-(3-(trifluoromethyl)phenylethylidene)-benzohydrazide

General procedure X was employed to give a mixture of isomers of the title compound as a colourless crystalline solid in 49% yield.

¹H-NMR (300 MHz, DMSO-d₆) δ 12.51 (s, 1H, major isomer), 12.41 (s, 1H), 12.27 (s, 1H, minor isomer), 8.63 (s, 1H, major isomer), 8.59 (s, 1H, minor isomer), 8.03 (bs, 1H), 7.84-7.40 (m, 7H).

### Example 35

### 4-tert-Butyl-N′-(2-(hydroxyimino)-1-(3-(trifluoromethyl)phenyl)ethylidene)benzohydrazide

General procedure X was employed to give the title compound as a colourless crystalline solid in 43% yield.

¹H-NMR (300 MHz, DMSO-d₆) δ 13.19 (s, 1H), 12.65 (s, 1H), 8.62 (s, 1H), 8.09-8.07 (m, 2H), 7.83 (t, 3H), 7.70 (t, 1H), 7.58 (d, 2H), 1.33 (s, 9H).

### Example 36

### 2,5-Dichloro-N′-(2-(hydroxyimino)-1-phenylethylidene)benzohydrazide

General procedure X was employed to give a mixture of isomers of the title compound as a colourless crystalline solid in 20% yield.

¹H-NMR (300 MHz, DMSO-d₆) δ 12.50 (s, 1H, major isomer), 12.44 (s, 1H, minor isomer), 12.33 (s, 1H, major isomer), 12.18 (s, 1H, minor isomer), 8.52 (s, 1H, major isomer), 8.50 (s, 1H, minor isomer), 7.80-7.57 (m, 4H), 7.46-7.32 (m, 4H).

### Example 37

### N′-(2-(Hydroxyimino)-1-(3-(trifluoromethyl)phenyl)ethylidene)-4-phenyl-benzohydrazide

General procedure X was employed to give the title compound as a colourless crystalline solid in 26% yield.

¹H-NMR (300 MHz, DMSO-d₆) δ 13.19 (s, 1H), 12.69 (s, 1H), 8.65 (s, 1H), 8.10-7.94 (m, 4H), 7.84-7.68 (m, 5H), 7.55-7.40 (m, 4H).

### Example 38

### 3-Bromo-N′-(2-(hydroxyimino)-1-(pyridin-2-yl)ethylidene)benzohydrazide

General procedure X was employed to give the title compound as a yellow crystalline solid in 42% yield.

¹H-NMR (300 MHz, DMSO-d₆) δ 13.51 (s, 1H), 12.73 (s, 1H), 8.87 (s, 1H), 8.63 (d, 1H), 8.05-8.06 (m, 2H), 7.99-7.84 (m, 3H), 7.54 (t, 1H), 7.47 (t, 1H), 2.50 (s, 3H).

### Example 39

### 3-Bromo-N′-(1-(2-bromophenyl)-2-(hydroxyimino)ethylidene)benzo-hydrazide

General procedure X was employed (using the intermediate from Preparation 5) to give the title compound as a beige powder in 8% yield.

¹H-NMR (300 MHz, DMSO-d₆) δ 12.37 (s, 1H), 12.22 (s, 1H), 8.55 (s, 1H), 8.09-8.03 (m, 1H), 7.92-7.75 (m, 2H), 6.69 (d, 1H), 7.53-7.34 (m, 4H).

### Example 40

### N′-(2-(Hydroxyimino)-1-phenylethylidene)-4-(trifluoromethyl)benzohydrazide

General procedure X was employed to give the title compound as a white crystalline solid in 74% yield.

¹H-NMR (300 MHz, DMSO-d₆) δ 13.09 (s, 1H), 12.58 (s, 1H), 8.54 (s, 1H), 8.08 (d, 2H), 7.91 (d, 2H), 7.78-7.75 (m, 2H), 7.50-7.40 (m, 3H).

### Example 41

### 3-Hydroxy-N′-(2-(hydroxyimino)-1-phenylethylidene)benzohydrazide

General procedure X was employed to give the title compound as a beige crystalline solid in 8% yield.

1H-NMR (300 MHz, DMSO-d₆) δ 13.1 (s, 1H), 12.60 (s, 1H), 9.83 (s, 1H), 8.50 (s, 1H), 7.78-7.75 (m, 2H), 7.38-7.35 (m, 3H), 7.33-7.25 (m, 3H), 7.03 (dd, 1H).

### Example 42

### 3-Amino-N′-(2-(hydroxyimino)-1-phenylethylidene)benzohydrazide

General procedure X was employed to give the title compound as a beige crystalline solid in 18% yield.

¹H-NMR (300 MHz, DMSO-d₆) δ 13.15 (s, 1H), 12.60 (s, 1H), 8.51 (s, 1H), 7.54-7.52 (m, 2H), 7.47-7.44 (m, 3H), 7.38-7.25 (m, 3H), 7.05-7.02 (m, 1H).

### Example 43

### N′-(2-(Hydroxyimino)-1-phenylethylidene)-3-methylbenzohydrazide

General procedure X was employed to give the title compound as a light yellow powder in 53% yield.

1-H-NMR (300 MHz, DMSO-d₆) δ 13.12 (s, 1H), 12.63 (s, 1H), 8.52 (s, 1H), 7.78-7.67 (m, 4H), 7.46-7.44 (m, 5H), 2.43 (s, 3H).

### Example 44

### N′-(2-(Hydroxyimino)-1-(3-hydroxyphenyl)ethylidene)-2-methylbenzohydrazide

General procedure X was employed to give the title compound as a brown crystalline solid in 26% yield.

¹H-NMR (300 MHz, DMSO-d₆) δ 12.65 (s, 1H), 12.55 (s, 1H), 9.85 (s, 1H), 8.58 (s, 1H), 7.75-7.30 (m, 7H), 7.05-7.02 (m, 1H), 2.68 (s, 3H).

### Example 45

### 3-Bromo-N′-(2-(hydroxyimino)-1-(3-(methylsulfonamido)phenyl)ethylidene)benzohydrazide

General procedure X was employed (using the intermediate from Preparation 6) to give the title compound as a beige crystalline solid in 66% yield.

¹H-NMR (300 MHz, DMSO-d₆) δ 12.8 (s, 1H), 12.66 (s, 1H), 9.93 (s, 1H), 8.54 (s, 1H), 8.11 (s, 1H), 7.92-7.90 (m, 2H), 7.63-7.36 (m, 5H), 3.07 (s, 3H).

### Example 46

### 3-Bromo-1-(1-(3-bromophenyl)-2-(hydroxyimino ethylidene)benzohydrazide

General procedure X was employed to give the title compound as a white powder in 72% yield.

¹H-NMR (300 MHz, DMSO-d₆) δ 12.8 (s, 1H), 12.66 (s, 1H), 8.57 (s, 1H), 8.05 (t, 1H), 7.92 (s, 1H), 7.84 (d, 2H), 7.77 (d, 1H), 7.64 (d, 1H), 7.52 (t, 1H), 7.41 (t, 1H).

### Example 47

### 3-Bromo-N′-(1-(3-chlorophenyl)-2-(hydroxyimino)ethylidene)benzohydrazide

General procedure X was employed to give the title compound as a white powder in 12% yield.

¹H-NMR (300 MHz, DMSO-d₆) δ 12.8 (s, 1H), 12.60 (s, 1H), 8.58 (s, 1H), 8.05 (t, 1H), 7.85 (d, 2H), 7.78 (s, 1H), 7.71 (d, 1H), 7.54-7.48 (m, 3H).

### Example 48

### 4-Fluoro-N′-(2-(hydroxyimino)-1-phenylethylidene)benzohydrazide

General procedure X was employed to give the title compound as a yellow powder in 54% yield.

¹H-NMR (300 MHz, DMSO-d₆) δ 13.10 (s, 1H), 12.62 (s, 1H), 8.60 (s, 1H), 7.99-7.95 (m, 2H), 7.76 (bs, 2H), 7.52-7.25 (m, 5H).

### Example 49

### N′-(2-(Hydroxyimino)-1-phenylethylidene)-4-methoxybenzohydrazide

General procedure X was employed to give the title compound as a white powder in 25% yield.

¹H-NMR (300 MHz, DMSO-d₆) δ 12.8 (s, 1H), 12.62 (s, 1H), 8.51 (s, 1H), 7.89 (d, 2H), 7.78-7.75 (m, 2H), 7.52-7.44 (m, 3H), 7.08 (d, 2H), 3.86 (s, 3H).

### Example 50

### N′-(2-(Hydroxyimino)-1-phenylethylidene)-2-methoxybenzohydrazide

General procedure X was employed to give a mixture of isomers of the title compound as a colourless crystalline solid in 20% yield.

¹H-NMR (300 MHz, DMSO-d₆) δ 12.96 (s, 1H, minor isomer), 12.29 (s, 1H, minor isomer), 11.81 (s, 1H, major isomer), 10.94 (s, 1H, major isomer), 8.41 (s, 1H, minor isomer), 8.06 (d, 1H, minor isomer), 8.03 (s, 1H, major isomer), 7.92-7.05 (m, 9H), 3.98 (s, 3H).

### Example 51

### N′-(2-(Hydroxyimino)-1-phenylethylidene)-2-nitrobenzohydrazide

General procedure X was employed to give a mixture of isomers of the title compound as a colourless crystalline solid in 32% yield.

¹H-NMR (300 MHz, DMSO-d₆) δ 12.52 (s, 1H, major isomer), 12.43 (s, 1H, major isomer), 12.32 (s, 1H, minor isomer), 12.40 (s, 1H, minor isomer), 8.49 (s, 1H, major isomer), 8.48 (s, 1H, minor isomer), 8.22-8.17 (m, 1H), 7.92-7.89 (m, 1H), 7.87-7.70 (m, 3H), 7.47-7.45 (m, 1H), 7.35-7.27 (m, 3H).

### Example 52

### N′-(2-(Hydroxyimino)-1-phenylethylidene)-4-methylbenzohydrazide

General procedure X was employed to give the title compound as a white powder in 18% yield.

¹H-NMR (300 MHz, DMSO-d₆) δ 13.2 (s, 1H), 12.64 (s, 1H), 8.52 (s, 1H), 7.78 (d, 2H), 7.77-7.73 (m, 2H), 7.47-7.45 (m, 3H), 7.37 (d, 2H), 2.41 (s, 3H).

### Example 53

### N′-(2-(Hydroxyimino)-1-phenylethylidene)-2-fluorobenzohydrazide

General procedure X was employed to give the title compound as an off white cotton in 27% yield.

¹H-NMR (300 MHz, DMSO-d₆) δ 12.78 (bs, 1H), 12.50 (s, 1H), 8.48 (s, 1H), 7.88-7.35 (m, 9H).

### Example 54

### N′-(2-(Hydroxyimino)-1-phenylethylidene)-4-bromobenzohydrazide

General procedure X was employed to give the title compound as yellow crystals in 55% yield.

¹H-NMR (300 MHz, DMSO-d₆) δ 13.09 (bs, 1H), 12.60 (s, 1H), 8.53 (s, 1H), 7.83 (d, 2H), 7.77-7.74 (m, 4H), 7.46-7.44 (m, 3H).

### Example 55

### N′-(2-(Hydroxyimino)-1-phenylethylidene)-2-aminobenzohydrazide

General procedure X was employed to give the title compound as yellow needles in 37% yield.

¹H-NMR (300 MHz, DMSO-d₆) δ 13.09 (s, 1H), 12.60 (s, 1H), 8.50 (s, 1H), 7.79-7.76 (m, 2H), 7.47-7.44 (m, 4H), 7.26 (t, 1H), 6.81 (d, 1H), 6.65 (bs, 2H), 6.61 (t, 1H).

### Example 56

### N′-(2-(Hydroxyimino)-1-phenylethylidene)-2-methylbenzohydrazide

General procedure X was employed to give the title compound as off white crystals in 40% yield.

¹H-NMR (300 MHz, DMSO-d₆) δ 12.47 (s, 1H), 12.41 (s, 1H), 8.49 (s, 1H), 7.77 (bs, 1H), 7.51-7.33 (m, 8H), 2.43 (s, 3H).

### Example 57

### N′-(2-(Hydroxyimino)-1-(2-methoxyphenyl)ethylidene)-3-bromobenzohydrazide

General procedure X was employed to give the title compound as a white powder in 44% yield.

¹H-NMR (300 MHz, DMSO-d₆) δ 12.81 (s, 1H), 12.36 (s, 1H), 8.25 (s, 1H), 8.05 (bs, 1H), 7.84 (d, 2H), 7.54-7.35 (m, 3H), 7.11 (d, 1H), 7.02 (t, 1H).

### Example 58

### N′-(2-(Hydroxyimino)-1-phenylethylidene)-2,4-dichlorobenzohydrazide

General procedure X was employed to give the title compound as a white powder in 58% yield (isomer-ratio (A/B): 1/1).

¹H-NMR (300 MHz, DMSO-d₆) δ 12.53 (s, 1H), 12.45 (s, 1H), 12.37 (s, 1H), 12.27 (s, 1H), 8.52 (s, 1H), 8.49 (s, 1H), 7.80-7.69 (m, 3H), 7.60-7.54 (m, 1H), 7.46-7.42 (m, 3H), 7.36-7.32 (m, 1H).

### Example 59

### N′-(2-(Hydroxyimino)-1-phenylethylidene)-3,4-dimethoxybenzohydrazide

General procedure X was employed to give the title compound as a white powder in 71% yield.

¹H-NMR (300 MHz, DMSO-d₆) δ 13.90 (bs, 1H), 12.60 (s, 1H), 8.51 (s, 1H), 7.78-7.75 (m, 2H), 7.49-7.45 (m, 5H), 7.08 (d, 1H), 3.86 (s, 3H), 3.85 (s, 3H).

### Example 60

### N′-(2-(Hydroxyimino)-1-phenylethylidene)-2,5-dichlorobenzohydrazide

General procedure X was employed to give the title compound as a white powder in 24% yield (isomer-ratio (A/B): 1/1)

¹H-NMR (300 MHz, DMSO-d₆) δ 12.52 (s, 1H, form A), 12.45 (s, 1H, form B), 12.35 (s, 1H, form A), 12.19 (s, 1H, form B), 8.52 (s, 1H, form A), 8.50 (s, 1H, form B), 7.80 (bs, 1H, form A or B), 7.73-7.70 (m, 2H, form A+B), 7.67 (d, 1H, form A or B), 7.63-7.57 (m, 4H, form A+B), 7.47-7.42 (m, 5H, form A+B), 7.36-7.30 (m, 3H, form A+B).

### Example 61

### N′-(2-(Hydroxyimino)-1-phenylethylidene)-4-nitrobenzohydrazide

General procedure X was employed to give the title compound as yellow crystals in 31% yield.

¹H-NMR (300 MHz, DMSO-d₆) δ 13.06 (s, 1H), 12.58 (s, 1H), 8.54 (s, 1H), 8.35 (d, 2H), 8.12 (d, 2H), 7.75 (s, 1H), 7.50-7.38 (m, 4H).

### Example 62

### N′-(2-(Hydroxyimino)-1-m-tolylethylidene)-3-chlorobenzohydrazide

General procedure X was employed to give the title compound as a white powder in 41% yield.

¹H-NMR (300 MHz, DMSO-d₆) δ 12.58 (s, 1H), 8.52 (s, 1H), 7.92-7.88 (m, 2H), 7.81 (d, 1H), 7.72-7.51 (m, 4H), 7.38-7.24 (m, 1H), 2.36 (s, 3H).

### Example 63

### N′-(2-(Hydroxyimino)-1-(3-nitrophenyl)ethylidene)-3-chlorobenzohydrazide

General procedure X was employed to give the title compound as a white powder in 58% yield.

¹H-NMR (300 MHz, DMSO-d₆) δ 12.63 (s, 1H), 8.67 (s, 1H), 8.55 (bs, 1H), 8.32 (t, 1H), 8.20 (d, 1H), 7.94 (s, 1H), 7.90-7.88 (m, 1H), 7.83 (d, 1H), 7.79-7.69 (m, 2H), 7.62-7.52 (m, 1H).

### Example 64

### N′-(2-(Hydroxyimino)-1-phenylethylidene)-5-methyl-1H-pyrazole-3-carbohydrazide

General procedure X was employed to give the title compound as colourless crystals in 5% yield.

¹H-NMR (300 MHz, DMSO-d6) δ 13.05 (s, 1H), 13.01 (s, 1H), 12.55 (s, 1H), 8.40 (s, 1H), 7.75 (dd, 2H), 7.46-7.43 (m, 3H), 6.55 (s, 1H), 3.31 (s, 3H).

### Example 65

### 6-Methylpyridine-2-carboxylic acid (2-hydroxyimino-1-phenylethylidene)-hydrazide

General procedure X was employed to give the title compound as yellow crystals in 13% yield.

¹H-NMR (300 MHz, DMSO-d₆): 13.74 (s, 1H), 12.50 (s, 1H), 8.45 (s, 1H), 7.98-7.92 (m, 2H), 7.82-7.80 (m, 2H), 7.55 (dd, 1H), 7.49-7.47 (m, 3H), 2.50 (s, 3H).

### Example 66

### 6-Chloronicotinic acid (2-hydroxyimino-1-phenylethylidene)hydrazide

General procedure X was employed to give the title compound as a white powder in 73% yield.

¹H-NMR (300 MHz, DMSO-d₆): 12.92 (s, 1H), 12.58 (s, 1H), 8.89 (bs, 1H), 8.56 (s, 1H), 8.30-8.27 (m, 1H), 7.75-7.71 (m, 3H), 7.46 (bs, 3H).

### Example 67

### 3-Methoxybenzoic acid [2-hydroxyimino-1-(3-nitrophenyl)ethylidene]-hydrazide

General procedure X was employed to give the title compound as off-white crystals in 42% yield.

¹H-NMR (300 MHz, DMSO-d₆): 12.99 (s, 1H), 12.67 (s, 1H), 8.63 (s, 1H), 8.57 (bs, 1H), 8.29 (d, 1H), 8.21 (d, 1H), 7.75 (t, 1H), 7.51-7.43 (m, 3H), 7.23 (d, 1H), 3.32 (s, 3H).

### Example 68

### 6-Methylnicotinic acid (2-hydroxyimino-1-phenylethylidene)hydrazide

General procedure X was employed to give the title compound as white cotton crystals in 27% yield.

¹H-NMR (300 MHz, DMSO-d₆): 13.09 (s, 1H), 12.64 (s, 1H), 8.94 (bs, 1H), 8.53 (s, 1H), 8.14 (dd, 1H), 7.76 (bs, 2H), 7.45 (bs, 3H), 2.58 (s, 3H).

### Example 69

### 5-Bromonicotinic acid (2-hydroxyimino-1-phenylethylidene)hydrazide

General procedure X was employed to give the title compound as off white flakes in 42% yield.

¹H-NMR (300 MHz, DMSO-d₆): 12.67 (s, 1H), 12.55 (s, 1H), 9.00 (bs, 1H), 8.95 (bs, 1H), 8.57 (s, 1H), 8.48 (bs, 1H), 7.73 (bs, 2H), 7.46 (bs, 3H).

### Example 70

### 3-Methylbenzoic acid [1-(3-chlorophenyl)-2-hydroxniminoethylidene]-hydrazide

General procedure X was employed to give the title compound in 1.9% yield.

¹H-NMR (300 MHz, DMSO-d₆): 13.09 (0.7H, bs), 12.85 (0.3H, bs), 12.66 (1H, s), 8.56 (1H, s), 7.93-7.35 (8H, m), 2.43 (2.1H, s), 2.36 (0.9H, s).

### Example 71

### 3-Methoxybenzoic acid [1-(3-chlorophenyl)-2-hydroxniminoethylidene]-hydrazide

General procedure X was employed to give the title compound in 2.3% yield.

¹H-NMR (300 MHz, DMSO-d₆): 13.03-12.97 (1H, m), 12.63 (1H, s), 8.55 (1H, s), 7.94-7.18 (8H, m), 3.85 (1.8H, s), 3.80 (1.2H, s).

### Example 72

### 3-Chlorobenzoic acid [1-(3-fluorophenyl)-2-hydroxyiminoethylidene]-hydrazide

General procedure X was employed to give the title compound in 15% yield.

¹H-NMR (300 MHz, DMSO-d₆): 13.32 (0.2H, bs), 12.91 (0.8H, bs), 12.62 (1H, s), 8.57 (1H, s), 7.92-7.16 (8H, m).

### Example 73

### 3-Fluorobenzoic acid [1-(3-chlorophenyl)-2-hydroxyiminoethylidene]-hydrazide

General procedure X was employed to give the title compound in 2.8% yield.

¹H-NMR (300 MHz, DMSO-d₆): 12.98 (1H, bs), 12.65 (1H, s), 8.57 (1H, s), 7.93-7.46 (8H, m).

### Example 74

### 3-Chloro-N′-(2-(hydroxyimino)-1-phenylpropylidene)benzohydrazide

General procedure X was employed to give the title compound.

¹H-NMR (300 MHz, DMSO-d6): 12.21 (bs, 1H), 12.09 (bs, 1H), 7.82 (s, 1H), 7.75 (d, J=7 Hz, 1H), 7.69 (d, J=7 Hz, 1H), 7.62-7.52 (m, 3H), 7.51-7.43 (m, 3H), 1.97 (s, 3H).

### Example 75

### N′-(2-(Benzyloxyimino)-1-phenylethylidene)benzohydrazide

To a solution of benzoic acid (2-hydroxyimino-1-phenylethylidene)-hydrazide (5.0 mmol) and KOH (6 mmol) in DMSO (5 mL) was added benzyl bromide. The reaction was stirred at 45° C. for 6 days. Water was added and the mixture was extracted with CH₂Cl₂. The combined extracts were washed with NaOH (2M) and water. The organic phase was dried (Na₂SO₄) and concentrated in vacuo to give a yellow oil. The oil was dissolved in a small volume of EtOH and water was added until precipitation occurred. The crystals were filtered off and recrystallised (EtOH/H₂O) to give 70 mg of the title compound as a beige powder in 4% yield.

¹H-NMR (DMSO-d₆): δ 12.59 (bs, 1H), 8.69 (s, 1H), 7.81 (d, 2H), 7.75 (bs, 2H), 7.63 (t, 1H), 7.54 (t, 2H), 7.44-7.32 (m, 8H), 5.36 (s, 2H).

MS (M⁺+H) m/z=358.

### Example 76

### N′-(2-(Ethoxyimino)-1-phenylethylidene)benzohydrazide

The title compound was prepared in accordance with Example 75 using ethyl bromide instead of benzyl bromide. The procedure furnished the title compound (127 mg, 14% yield) as colourless crystals.

¹H-NMR (DMSO-d₆): δ 12.78 (bs, 1H), 8.60 (s, 1H), 7.89 (d, 2H), 7.77 (bs, 2H), 7.65 (t, 1H), 7.58 (t, 2H), 7.46 (t, 3H), 4.33 (q, 2H), 1.31 (t, 3H).

MS (M⁺+H) m/z=296.

### Example 77

### 3-Chloro-N′-(2-(hydroxyimino)-1-(3-(trifluoromethyl)phenyl)ethylidene)benzohydrazide

General procedure Y was employed using 2.3 mmol of the relevant hydrazide to give the title compound colourless crystals (410 mg, 48% yield).

¹H-NMR (DMSO-d₆): δ 12.80 (bs, 1H), 12.60 (s, 1H), 8.64 (s, 2H), 8.05 (bs, 1H), 7.93 (t, 1H), 7.83-7.81 (m, 2H), 7.71-7.68 (m, 2H), 7.59 (t, 1H). MS (M⁺+H) m/z=370.

### Example 78

### 3-Nitro-N′-(2-(hydroxyimino)-1-(3-chlorophenyl)ethylidene)benzohydroazide

General procedure Y was employed to give the title compound as a colourless powder (237 mg, 68% yield).

¹H-NMR (DMSO-d₆): δ 12.85 (bs, 1H), 12.57 (s, 1H), 8.69 (t, 1H), 8.60 (s, 1H), 8.46 (d, 1H), 8.28 (d, 1H), 7.85 (t, 1H), 7.75-7.65 (m, 2H), 7.53-7.40 (m, 2H).

MS (M⁺+H) m/z=347.

### Example 79

### 4-Nitro-N′-(2-(hydroxyimino)-1-(3-chlorophenyl)ethylidene)benzohydroazide

General procedure Y was employed to give the title compound as a yellow powder (254 mg, 73% yield).

1H-NMR (DMSO-d₆): δ 13.03 (bs, 1H), 12.61 (s, 1H), 8.59 (s, 1H), 8.35 (d, 2H), 8.12 (d, 2H), 7.80 (bs, 1H), 7.73 (bs, 1H), 7.50-7.46 (m, 2H).

MS (M⁺+H) m/z=347.

### Example 80

### 3-(2-Hydroxyimino-1-phenylethylidenehydrazinocarbonyl)-N,N-dimethylbenzenesulfonamide

General procedure Y was employed using 2.1 mmol of the relevant hydrazide to give the title compound as colourless crystals (320 mg, 41% yield).

¹H-NMR (DMSO-d₆): δ 12.84 (bs, 1H), 12.56 (s, 1H), 8.57 (s, 1H), 8.21 (bs, 1H), 8.15 (d, 1H), 8.00-7.71 (m, 4H), 7.51-7.43 (m, 3H), 2.65 (bs, 6H).

MS (M⁺+H) m/z=375.

### Example 81

### 3-(2-Hydroxyimino-1-(3-bromophenyl)ethylidenehydrazinocarbonyl)-N,N-dimethylbenzenesulfonamide

General procedure Y was employed using 2.1 mmol of the relevant hydrazide to give the title compound as colourless crystals (150 mg, 16% yield).

¹H-NMR (DMSO-d₆): δ 12.92 (bs, 1H), 12.54 (s, 1H), 8.55 (s, 1H), 8.23 (t, 1H), 8.15 (d, 1H), 8.00-7.61 (m, 5H), 7.43 (bs, 1H), 2.65 (bs, 6H).

MS (M⁺+H) m/z=453.

### Example 82

### 3-(Trifluoromethyl)-N′-(2-(hydroxyimino)-1-phenylethylidene)benzohydrazide

The title compound was prepared in accordance with the procedures described herein using 50 mg (245 μmol) of the relevant hydrazide to yield the title compound (13.4 mg, 16%).

¹H-NMR (400 MHz, DMSO-d₆) δ 12.89 (bs, 1H), 12.59 (bs, 1H), 8.56 (s, 1H), 8.22 (m, 1H), 8.15 (d, J=8 Hz, 1H), 8.02 (d, J=8 Hz, 1H), 7.82 (d, J=8 Hz, 1H), 7.80-7.70 (m, 2H), 7.50-7.40 (m, 3H).

MS (M⁺+H) m/z=336.

### Example 83

### 1,3,5-Trimethyl-1H-pyrazole-4-carboxylic acid (2-hydroxyimino-1-phenylethylidene)hydrazide

The title compound was prepared in accordance with the procedures described herein using 42 mg scale (250 μmol) of the relevant hydrazide to yield the title compound (31 mg, 42%).

¹H-NMR (270 MHz, DMSO-d₆) δ 12.42 (bs, 1H), 12.40 (bs, 1H), 8.44 (s, 1H), 7.76-7.10 (m, 2H), 7.47-7.40 (m, 3H), 3.70 (s, 3H), 2.41 (s, 3H), 2.30 (s, 3H).

MS (M⁺+H) m/z=300.

### Example 84

### 2-Nitro-N′-(2-(hydroxyimino)-1-(3-chlorophenyl)ethylidene)benzohydroazide

General procedure Y was employed using 1.0 mmol of the relevant hydrazide to give the title compound as a white powder (46% yield).

¹H-NMR (DMSO-d₆) δ 12.52 (s, 1H), 12.43 (s, 1H), 8.52 (s, 1H), 8.21 (d, 1H), 7.90 (t, 1H), 7.81-7.75 (m, 2H), 7.71 (d, 1H), 7.40 (dt, 1H), 7.35-7.28 (m, 2H).

MS (M⁺+H) m/z=347.

### Example 85

### 5-Bromonicotinic acid (2-hydroxyimino-1-(3-chlorophenyl)ethylidene)-hydrazide

General procedure Y was employed using 2.0 mmol of the relevant hydrazide to give the title compound as a white powder (39%).

¹H-NMR (DMSO-d₆) δ 12.62 (s, 1H), 12.58 (s, 1H), 8.97 (d, 2H), 8.61 (s, 1H), 8.49 (t, 1H), 7.77-7.70 (m, 2H), 7.53-7.48 (m, 2H).

MS (M⁺+H) m/z=381.

### Example 86

### 3,4-Dichloro-N′-(2-(hydroxyimino)-1-(3-chlorophenyl)ethylidene)benzohydrazide

General procedure Y was employed using 1.0 mmol of the relevant hydrazide to give the title compound as a white powder (57%).

¹H-NMR (DMSO-d₆) δ 12.68 (bs, 1H), 12.66 (s, 1H), 8.58 (s, 1H), 8.12 (d, 1H), 7.84-7.73 (m, 3H), 7.53-7.46 (m, 3H).

MS (M⁺+H) m/z=370.

### Example 87

### 3-Nitro-N′-(2-(hydroxyimino)-1-(3-nitrophenyl)ethylidene)benzohydrazide

General procedure Y was employed using 2.0 mmol of the relevant hydrazide to give the title compound as a white powder (66%).

¹H-NMR (DMSO-d₆) δ 12.79 (bs, 1H), 12.60 (s, 1H), 8.70-8.69 (m, 2H), 8.55 (bs, 1H), 8.46 (d, 1H), 8.30 (d, 2H), 8.19 (bs, 1H), 7.87 (t, 1H), 7.75 (t, 1H).

MS (M⁺+H) m/z=358.

### Example 88

### 3-Chloro-N′-(2-(hydroxyimino)-1-(3-cyanophenyl)ethylidene)benzohydroazide

General procedure Y was employed using 1.0 mmol of the relevant hydrazide to give the title compound as a white powder in 67% yield.

¹H-NMR (DMSO-d₆) δ 12.85 (bs, 1H), 12.62 (s, 1H), 8.63 (s, 1H), 8.16 (bs, 1H), 8.08 (bs, 1H), 7.94-7.90 (m, 2H), 7.82 (d, 1H), 7.73-7.57 (m, 3H).

MS (M⁺+H) m/z=327.

### Example 89

### 3-Chloro-N′-(2-(hydroxyimino)-1-(4-cyanophenyl)ethylidene)benzohydrazide

General procedure Y was employed using 1.0 mmol of the relevant hydrazide to give the title compound as a white powder (29%).

¹H-NMR (DMSO-d₆) δ 12.90 (bs, 1H), 12.64 (s, 1H), 8.61 (s, 1H), 7.97-7.86 (m, 4H), 7.82 (d, 1H), 7.72 (d, 1H), 7.59 (d, 2H).

MS (M⁺+H) m/z=327.

### Example 90

### 3-(2-Hydroxyimino-1-phenylethylidenehydrazinocarbonyl)-N-methyl benzenesulfonamide

General procedure Y was employed using 1.3 mmol of the relevant hydrazide to give the title compound as colourless crystals (34%).

¹H-NMR (DMSO-d₆) δ12.99 (bs, 1H), 12.56 (s, 1H), 8.55 (s, 1H), 8.31 (s, 1H), 8.09 (d, 1H), 8.01 (d, 1H), 7.81-7.77 (m, 1H), 7.40-7.50 (m, 5H), 2.45-2.40 (m, 4H).

MS (M⁺+H) m/z=361.

### Example 91

### 3,5-Dichloro-N′-(2-(hydroxyimino)-1-(3-chlorophenyl)ethylidene)benzohydrazide

General procedure Y was employed using 1.0 mmol of the relevant hydrazide to give the title compound as a white powder (80%).

¹H-NMR (DMSO-d) δ 12.53 (s, 1H), 12.40 (s, 1H), 8.59 (s, 1H), 7.88 (bs, 2H), 7.68 (bs, 1H), 7.62 (bs, 1H), 7.53-7.46 (m, 3H).

MS (M⁺+H) m/z=370.

### Example 92

### 4-N,N-Dimethylamino-N′-(2-(hydroxyimino)-1-(3-chlorophenyl)ethylidene)benzohydrazide

General procedure Y was employed using 2.8 mmol of the relevant hydrazide to give the title compound as yellow crystals (18% yield).

¹H NMR (DMSO-d₆) δ 13.1 (bs, 1H), 12.6 (bs, 1H), 8.53 (s, 1H), 7.82-7.73 (m, 4H), 6.78-6.76 (m, 2H), 7.47 (d, 2H), 3.03 (s, 6H).

MS (M⁺+H) m/z=345.

### Example 93

### 3,5-Dibromo-N′-(2-(hydroxyimino)-1-(3-chlorophenyl)ethylidene)benzohydrazide

General procedure Y was employed using 1.4 mmol of the relevant hydrazide to give the title compound as colourless crystals (51%).

¹H NMR (DMSO-d₆) δ 12.51 (bs, 1H), 12.32 (bs, 1H), 8.60 (s, 1H), 8.11 (bs, 1H), 8.04-8.01 (m, 2H), 7.77-7.68 (m, 2H), 7.53-7.47 (m, 2H).

MS (M⁺+H) m/z=458.

### Example 94

### 3-N,N-Dimethylamino-N′-(2-(hydroxyimino)-1-(3-chlorophenyl)ethylidene)benzohydrazide

General procedure Y was employed using 1.7 mmol of the relevant hydrazide to give the title compound as colourless crystals (9%).

¹H NMR (DMSO-d₆) δ 13.05 (bs, 1H), 12.60 (bs, 1H), 8.53 (s, 1H), 7.81 (bs, 1H), 7.74 (d, 1H), 7.53-7.46 (m, 2H), 7.39 (t, 1H), 7.28 (bs, 1H), 7.19 (d, 1H), 7.09 (d, 1H), 3.01 (s, 6H).

MS (M⁺+H) m/z=345.

### Example 95

### 3,4-Dichloro-N′-(2-(hydroxyimino)-1-(3,4-dichlorophenyl)ethylidene)-benzohydrazide

General procedure Y was employed using 1.5 mmol of the relevant hydrazide to give the title compound as colourless crystals (38%).

¹H NMR (DMSO-d₆) δ 12.68 (bs, 1H), 12.59 (s, 1H), 8.60 (s, 1H), 8.12 (s, 1H), 7.90 (bs, 1H), 7.81-7.71 (m, 4H).

MS (M⁺+H) m/z=404.

### Example 96

### 4-Methylnicotinic acid (2-hydroxyimino-1-(3-chlorophenyl)ethylidene)-hydrazide

General procedure Y was employed using 0.8 mmol of the relevant hydrazide to give the title compound as yellow crystals (36%).

¹H NMR (DMSO-d₆) δ 12.72 (bs, 1H), 12.65 (bs, 1H), 8.97 (d, 1H), 8.73-8.60 (m, 2H), 8.25 (s, 1H), 7.78-7.70 (m, 2H), 7.56-7.44 (m, 2H), 2.45 (s, 3H).

MS (M⁺+H) m/z=317.

### Example 97

### 3-Chloro-N′-(2-(hydroxyimino)-1-(3-chloro-4-fluorophenyl)ethylidene)-benzohydrazide

General procedure Y was employed using 1.0 mmol of the relevant hydrazide to give the title compound as yellow crystals (18%).

¹H NMR (DMSO-d₆) δ 12.79 (bs, 1H), 12.62 (bs, 1H), 8.59 (s, 1H), 7.92 (t, 1H), 7.83-7.45 (m, 6H).

MS (M⁺+H) m/z=354.

### Example 98

### Pyrazine-2-carboxylic acid (1-(3-chlorophenyl)-2-hydroxyiminoethylidene)hydrazide

General procedure Y was employed using 3.3 mmol of the relevant hydrazide to give the title compound as yellow crystals (6%).

¹H NMR (DMSO-d₆) δ 13.93 (s, 1H), 12.75 (s, 1H), 9.31 (s, 1H), 8.97 (d, 1H), 8.77 (s, 1H), 8.50 (s, 1H), 7.87 (bs, 1H), 7.79 (d, 1H), 7.55-7.49 (m, 2H).

MS (M⁺+H) m/z=304.

### Example 99

### 3-Chloro-N′-(2-(hydroxyimino)-1-(4-pyridyl)ethylidene)benzohydrazide

General procedure Y was employed using 1.0 mmol of the relevant hydrazide, followed by recrystallisation (from EtOH) to give the title compound (31%, 95 mg) as light brown crystals.

¹H-NMR (DMSO-d₆): δ 13.02 (bs, 0.4H), 12.73 (s, 1H), 8.77 (d, 2H), 8.67 (s, 1H), 7.95 (m, 3H), 7.83 (d, 1H), 7.74 (dd, 1H), 8.10 (t, 1H).

MS (M⁺+H) m/z=303.

### Example 100

### 6-Chloropicolinic acid (2-hydroxyimino-1-(3-chlorophenyl)ethylidene)hydrazide

General procedure Y was employed using 1.5 mmol of the relevant hydrazide to give the title compound as colourless crystals (6% yield).

¹H NMR (DMSO-d₆) δ 12.65 (bs, 1H), 12.59 (bs, 1H), 8.63 (d, 1H), 8.60 (s, 1H), 7.93 (bs, 1H), 7.80-7.70 (m, 3H), 7.55-7.49 (m, 2H).

MS (M⁺+H) m/z=337.

### Example 101

### 5-Methylnicotinic acid (2-hydroxyimino-1-(3-chlorophenyl)ethylidene)hydrazide

General procedure Y was employed using 1.0 mmol of the relevant hydrazide to give the title compound as colourless crystals (4%).

¹H-NMR (DMSO-d₆) δ 12.44 (bs, 1H), 12.37 (bs, 1H), 8.69-8.47 (m, 3H), 7.78-7.70 (m, 1H), 7.51-7.30 (m, 4H), 2.22 (s, 3H).

MS (M⁺+H) m/z=317.

### Example 102

### 3-Chloro-N′-(2-(hydroxyimino)-1-(4-morpholinophenyl)ethylidene)benzohydrazide

General procedure Y was employed using 1.0 mmol of the relevant hydrazide to give the title compound as yellow crystals (70%).

¹H-NMR (DMSO-d₆) δ 12.84 (bs, 1H), 12.51 (bs, 1H), 8.49 (s, 1H), 7.89 (bs, 1H), 7.79 (d, 1H), 7.69 (bs, 3H), 7.59 (dd, 1H), 6.99 (d, 2H), 3.75 (t, 4H), 3.21 (bs, 4H).

MS (M⁺+H) m/z=387.

### Example 103

### 3-Chloro-4-N,N-dimethylamino-N′-(2-(hydroxyimino)-1-(3-chlorophenyl)ethylidene)benzohydrazide

General procedure Y was employed using 1.0 mmol of the relevant hydrazide to give the title compound as colourless crystals in 41% yield.

¹H-NMR (DMSO-d₆) δ 12.88 (bs, 1H), 12.60 (s, 1H), 8.56 (s, 1H), 7-89 (d, 1H), 7.79-7.71 (m, 3H), 7.53-7.47 (m, 2H), 7.19 (d, 1H), 2.87 (s, 6H).

MS (M⁺+H) m/z=379.

### Example 104

### 6-Hydroxynicotinic acid [1-(3-chlorophenyl)-2-hydroxniminoethylidene]-hydrazide

General procedure Y was employed to give the title compound as colourless crystals (170 mg, 27%).

¹H-NMR (DMSO-d₆) δ 12.55 (s, 1H), 12.52 (bs, 1H), 8.55 (s, 1H), 8.14 (bs, 1H), 8.84 (dd, 1H), 7.76 (bs, 1H), 6.68 (d, 1H), 7.51-7.43 (m, 3H), 6-41 (d, 1H).

MS (M⁺+H) m/z=319.

### Example 105

### 5-Chloro-2-N,N-dimethylamino-N′-(2-(hydroxyimino)-1-(3-chlorophenyl)ethylidene)benzohydrazide

General procedure Y was employed using 1.0 mmol of the relevant hydrazide to give the title compound as off-white powder (137 mg, 36%).

¹H-NMR (DMSO-d₆): δ 13.51 (s, 1H), 12.27 (s, 1H), 8.41 (s, 1H), 7.80 (m, 1H), 7.74 (m, 2H), 7.51 (m, 3H), 7.31 (d, 1H), 2.74 (s, 6H).

MS (M⁺+H) m/z=379.

### Example 106

### 3-Chloro-N′-(2-(hydroxyimino)-1-(4-dimethylamino-phenyl)ethylidene)benzohydrazide

General procedure Y was employed to give the title compound (84 mg, 24%) as brown crystals.

¹H-NMR (DMSO-d₆): δ 12.81 (bs, 1H), 12.48 (s, 1H), 8.47 (s, 1H), 7.88 (m, 1H), 7.79 (dt, 1H), 7.67 (m, 3H), 7.57 (t, 1H), 6.75 (d, 2H), 2.97 (s, 6H).

MS (M⁺+H) m/z=345.

### Example 107

### 3-Chloro-N′-(2-(hydroxyimino)-1-(3-bromophenyl)ethylidene)benzohydroazide

General procedure Y was employed to give the title compound (275 mg, 72%) as white crystals.

¹H-NMR (DMSO-d₆): δ 12.83 (bs, 1H), 12.60 (s, 1H), 8.57 (s, 1H), 7.96-7.39 (m, 8H).

MS (M⁺+H) m/z=380.

### Example 108

### 3-Chloro-Y-(2-(hydroxyimino)-1-(3-chlorophenyl)ethylidene)benzohydrazide

General procedure Y was employed using 1.2 mmol of the relevant hydrazide to give the title compound (184 mg, 55%) as white crystals.

¹H-NMR (DMSO-d₆): δ 12.85 (bs, 1H), 12.60 (s, 1H), 8.57 (s, 1H), 7.92 (s, 1H), 7.81 (bd, 2H), 7.71 (bd, 2H), 7.62-7.45 (m, 3H).

MS (M⁺+H) m/z==336.

### Example 109

### 3-(Trifluoromethyl)-N′-(2-(hydroxyimino)-1-(3-chlorophenyl)ethylidene)benzohydrazide

General procedure Y was employed to give the title compound (144 mg, 39%) as white crystals.

¹H-NMR (DMSO-d₆): δ 12.82 (bs, 1H), 12.58 (s, 1H), 8.59 (s, 1H), 8.22 (s, 1H), 8.16 (d, 1H), 8.02 (d, 1H), 7.86-7.68 (m, 3H), 7.54-7.46 (m, 2H).

MS (M⁺+H) m/z=370.

### Example 110

### 3-Chloro-N′-(2-(ethoxyimino)-1-phenylethylidene)benzohydrazide

3-Chloro-N′-(2-(hydroxyimino)-1-phenylethylidene)benzohydrazide (300 mg, 1.0 mmol; see Example 4) was suspended in NaOH (2.4 mL, 0.4 M), toluene (3 mL), tetrabutylammonium bromide (16 mg, 0.04 mmol) and bromoethane (82 μL, 1.1 mmol). The reaction was refluxed overnight. The phases were separated and the organic phase was dried (Na₂SO₄) and the solvent removed in vacuo. The residue was dissolved in EtOH, water was added and the resulting precipitate was filtered off to give the title compound (23 mg, 7%) as white solid.

¹H-NMR (DMSO-d₆): δ 12.62 (bs, 1H), 8.60 (s, 1H), 7.91 (s, 1H), 7.84 (d, 1H), 7.79-7.67 (m, 3H), 7.59 (t, 1H), 7.45 (bs, 3H), 4.32 (q, 2H), 1.30 (t, 3H).

MS (M⁺+H) m/z=330.

### Example 111

### 6-Methylpicolinic acid (2-hydroxyimino-1-(3-chlorophenyl)ethylidene)hydrazide

General procedure Y was employed using 1.0 mmol of the hydrazide to give the title compound (201 mg, 63%) as a light yellow solid.

¹H-NMR (DMSO-d₆): δ 13.78 (s, 1H), 12.55 (s, 1H), 8.47 (s, 1H), 7.96-7.92 (m, 2H), 7.86 (d, 1H), 7.77 (dt, 1H), 7.56-7.48 (m, 3H), 2.66 (s, 3H).

MS (M⁺+H) m/z=317.

### Example 112

### 3-Chloro-N′-(2-(hydroxyimino)-1-(benzo[1,3]dioxol-5-yl)ethylidene)-benzohydrazide

General procedure Y was employed using 1.0 mmol of the hydrazide to give a grey/yellow solid, which was washed with hot toluene to give the title product (203 mg, 59%) as a very insoluble grey/yellow solid.

¹H-NMR (DMSO-d₆): δ 12.87 (bs, 1H), 12.55 (s, 1H), 8.49 (s, 1H), 7.90 (s, 1H), 7.79 (d, 1H), 7.71-7.68 (m, 1H), 7.64-7.59 (m, 1H), 7.37-7.29 (m, 1H), 7.05-6.94 (m, 2H), 6.12-6.06 (m, 2H).

MS (M⁺+H) m/z=346.

### Example 113

### 2-Methylnicotinic acid (2-hydroxyimino-1-(3-chlorophenyl)ethylidene)-hydrazide

General procedure Y was employed to give a product, which was recrystallised (from EtOH/H₂O) to give the title compound as white cotton-like crystals.

¹H-NMR (DMSO-d₆): δ 12.41 (bs, 1H), 12.34 (bs, 1H), 8.66-8.50 (m, 2H), 7.97-7.71 (m, 2H), 7.51-7.27 (m, 4H), 2.59 (bs, 2H), 2.46 (bs, 1H).

MS (M⁺+H) m/z=317.

### Example 114

### 3-Chloro-N′-(2-(ethoxyimino)-1-(3-chlorophenyl)ethylidene)benzohydroazide

To a solution of 3-chloro-N′-(2-(hydroxyimino)-1-(3-chlorophenyl)ethylidene)benzohydrazide (see Example 108) in DMF at RT was added bromoethane and K₂CO₃. The mixture was stirred for 15 min, resulting in a yellow suspension. EtOAc was added and the organic phase was washed with NaOH (2M) and CaCl₂ (aq., saturated), water and NaCl (aq., sat.). The organic phase was dried (Na₂SO₄) and the solvent removed in vacuo to give a semisolid residue. Recrystallisation (from EtOH/H₂O) gave the title compound (15 mg, 4%) as an off-yellow solid.

¹H-NMR (DMSO-d₆): δ 12.66 (bs, 1H), 8.65 (s, 1H), 7.92 (t, 1H), 7.85 (d, 1H), 7.80 (s, 1H), 7.72 (d, 2H), 7.61 (t, 1H), 7.55-7.51 (m, 1H), 7.49 (t, 1H), 4.32 (q, 2H), 1.31 (t, 3H).

MS (M⁺+H) m/z=364.

### Example 115

### 3-Chloro-N′-(2-(hydroxyimino)-1-(quinoxalin-2-yl)ethylidene)benzohydrazide

General procedure Y was employed using 0.25 mmol of the relevant hydrazide to give the title compound (62 mg, 72%) as off-grayish crystals.

¹H-NMR (DMSO-d₆): δ 13.02 (bs, 1H), 9.57 (s, 1H), 8.72 (s, 1H), 8.15-8.11 (m, 2H), 7.92-7.85 (m, 5H), 7.70 (dd, 1H), 7.55 (t, 1H).

MS (M⁺+H) m/z=354.

### Example 116

### N′-(2-(Hydroxyimino)-1-(4-chlorophenyl)ethylidene)benzohydrazide

General procedure Y was employed to give the title compound as a white powder (75%).

¹H-NMR (300 MHz, DMSO-d₆) δ 13.16 (s, 1H), 12.65 (s, 1H), 8.54 (s, 1H), 7.90 (d, 2H), 7.80 (d, 2H), 7.66 (t, 1H), 7.58 (d, 2H), 7.52 (d, 2H)

MS (M⁺+H) m/z=302.

### Example 117

### 3-Nitro-Y-(2-(hydroxyimino)-1-phenylethylidene)benzohydrazide

General procedure Y was employed to give the title compound as white crystals (79%).

¹H-NMR (300 MHz, DMSO-d₆) δ 12.92 (s, 1H), 12.55 (s, 1H), 8.69 (s, 1H), 8.57 (s, 1H), 8.46 (d, 1H), 8.28 (d, 1H), 7.85 (t, 1H), 7.75 (bs, 2H), 7.46 (bs, 3H).

MS (M⁺+H) m/z=313.

### Example 118

### N′-(2-(2-Methoxyethoxyimino)-1-phenylethylidene)benzohydrazide

General procedure Z was employed using the relevant oxime (535 mg, 2 mmol) and 2-bromoethylmethyl ether (340 μL, 3.6 mmol) to give the title compound (64 mg, 10%) as a white powder.

¹H-NMR (DMSO-d₆): δ 12.69 (bs, 1H), 8.63 (s, 1H), 7.90 (d, 2H), 7.77 (bs, 2H), 7.65 (t, 1-H), 7.57 (t, 2H), 7.46 (t, 3H), 4.41 (t, 2H), 3.65 (t, 2H), 3.25 (s, 3H).

MS (M⁺+H) m/z=326.

### Example 119

### N′-(2-(Isopentoxyimino)-1-phenylethylidene)benzohydrazide

General procedure Z was employed using the relevant oxime (535 mg, 2 mmol) and 1-bromo-3-methylbutane (431 μL, 3.6 mmol) to give the title compound (401 mg, 59%) as white crystals.

¹H-NMR (DMSO-d₆): δ 12.76 (bs, 1H), 8.60 (s, 1H), 7.89 (d, 2H), 7.77 (bs, 2H), 7.65 (t, 1H), 7.57 (t, 2H), 7.46 (t, 3H), 4.32 (t, 2H), 1.79-1.66 (m, 1H), 1.59 (q, 2H), 0.89 (d, 6H).

MS (M⁺+H) m/z=338.

### Example 120

### N′-(2-(Propoxyimino)-1-phenylethylidene)benzohydrazide

General procedure Z was employed using the relevant oxime (535 mg, 2 mmol) and 1-bromopropane (330 μL, 3.6 mmol) to give the title compound (313 mg, 51%) as white flakes.

¹H-NMR (DMSO-d₆): δ 12.80 (bs, 1H), 8.61 (s, 1H), 7.89 (d, 2H), 7.77 (d, 2H), 7.65 (t, 1H), 7.57 (t, 2H), 7.46 (t, 3H), 4.24 (t, 2H), 1.76-1.67 (m, 2H), 0.94 (t, 3H).

MS (M⁺+H) m/z=310.

### Example 121

### N′-(2-(Isobutoxyimino)-1-phenylethylidene)benzohydrazide

General procedure Z was employed using the relevant oxime (535 mg, 2 mmol) and 1-bromo-2-methylpropane (395 μL, 3.6 mmol) to give the title compound (201 mg, 59%) as white flakes.

¹H-NMR (DMSO-d₆): δ 12.80 (bs, 1H), 8.63 (s, 1H), 7.88 (d, 2H), 7.77 (bs, 2H), 7.66 (t, 1H), 7.57 (t, 2H), 7.46 (t, 3H), 4.07 (d, 2H), 2.09-1.95 (m, 1H), 0.93 (d, 6H).

MS (M⁺+H) m/z=324.

### Example 122

### 3-Chloro-N′-(2-(isopentoxyimino)-1-phenylethylidene)benzohydrazide

General procedure Z was employed using the relevant oxime (1.5 g, 5 mmol; see Example 4) and 1-bromo-3-methylbutane (1.07 mL, 9 mmol). Recrystallisation (from EtOH/H₂O) gave the title compound (793 mg, 43%) as yellow crystals.

¹H-NMR (DMSO-d₆): δ 12.65 (bs, 1H), 8.61 (s, 1H), 7.92 (bs, 1H), 7.84 (d, 1H), 7.79-7.69 (m, 3H), 7.59 (t, 1H), 7.47-7.43 (m, 3H). 4.31 (t, 2H), 1.79-1.66 (m, 1H), 1.59 (q, 2H), 0.90 (d, 6H).

MS (M⁺+H) m/z=372.

### Example 123

### 3-Chloro-N′-(2-(isobutoxyimino)-1-(3-chlorophenyl)ethylidene)benzohydroazide

General procedure Z was employed using the relevant oxime (666 mg, 2 mmol; see Example 108) and 1-bromo-2-methylpropane (395 μL, 3_{—}6 mmol). Recrystallisation (from EtOH/H₂O) gave the title compound (187 mg, 23%) as yellow solid.

¹H-NMR (DMSO-d₆): δ 12.68 (bs, 1H), 8.68 (s, 1H), 7.92 (t, 1H), 7.86-7-78 (m, 2H), 7.72 (d, 2H), 7.60 (t, 1H), 7.54-7.46 (m, 2H). 4.07 (d, 2H), 2.08-1.94 (m, 1H), 0.94 (d, 6H).

MS (M⁺+H) m/z=392.

### Example 124

### 3-Chloro-N′-(2-(methoxyimino)-1-phenylethylidene)benzohydrazide

3-Chloro-N′-(2-(hydroxyimino)-1-phenylethylidene)benzohydrazide (150 mg, 0.5 mmol; see Example 4) was dissolved in MeOH/CH₂Cl₂ (100 mL, 1:1), after which methyl iodide (155 μL, 2.5 mmol) and silver(I)oxide (117 mg, 0.55 mmol) were added. The suspension was stirred for 5 h, after which additional methyl iodide (155 μL, 2.5 mmol) was added. The reaction was stirred at RT for 4 days. The mixture was filtered through Celite® and concentrated to dryness. Precipitation by dissolving the residue in EtOH and adding water gave the title compound (59 mg, 37%) as white crystals.

¹H-NMR (DMSO-d₆): δ 12.63 (bs, 1H), 8.61 (s, 1H), 7.93 (s, 1H), 7.85 (d, 1H), 7.76-7.71 (m, 3H), 7.61 (t, 1H), 7.46 (t, 3H), 4.07 (s, 3H).

MS (M⁺+H) m/z=316.

### Example 125

### 3-Chloro-N′-(2-(propoxyimino)-1-(3-chlorophenyl)ethylidene)benzohydroazide

To a solution of 3-chloro-N′-(2-(hydroxyimino)-1-(3-chlorophenyl)-ethylidene)benzohydrazide (672 mg, 2.0 mmol; see Example 108) in DMF (20 mL) at RT was added bromopropane (0.2 mL, 2.2 mmol) and potassium carbonate (180 mg, 1.3 mmol). The suspension was stirred for 30 min resulting in a yellow suspension. EtOAc was added, the organic phase was washed with 3 portions of NaOH (2M) and 2 portions of CaCl₂ (aq., sat.), water and NaCl (aq., sat.). The organic phase was dried (Na₂SO₄) and the solvent removed in vacuo. The residue was dissolved in EtOH. Addition of water gave a precipitate, which was collected and recrystallised (from EtOH/H₂O) to give the title compound (62 mg, 8%) as an off-yellow solid. ¹H-NMR (DMSO-d₆): δ 12.68 (bs, 1H), 8.65 (s, 1H), 7.91 (bs, 1H), 7.83 (d, 1H), 7.79 (s, 1H), 7.72 (d, 2H), 7.60 (t, 1H), 7.53-7.46 (m, 2H), 4.23 (t, 2H), 1.76-1.67 (m, 2H), 0.94 (t, 3H).

MS (M⁺+H) m/z=378.

### Example 126

### 3-Chloro-N′-(2-(hydroxyimino)-1-(4-ethylphenyl)ethylidene)benzohydroazide

General procedure Y was employed using 3-chlorobenzhydrazide (170 mg, 1.0 mmol) and 2-(4-ethylphenyl)-2-oxoacetaldehyde oxime (177 mg, 1.0 mmol). Recrystallisation (from EtOH/H₂O) gave the title compound (80 mg, 24%) as an off-white solid.

¹H-NMR (DMSO-d₆): δ 12.89 (bs, 1H), 12.56 (s, 1H), 8.51 (s, 1H), 7.90 (s, 1H), 7.80 (d, 1H), 7.69 (bs, 3H), 7.58 (t, 1H), 7.28 (d, 2H), 2.64 (q, 2H), 1.20 (t, 3H).

MS (M⁺+H) m/z=330.

### Example 127

### 3-Methylpicolinic acid (2-hydroxyimino-1-(3-chlorophenyl)ethylidene)-hydrazide

General procedure Y was employed using the appropriate hydrazide (151 mg, 1.0 mmol) and 2-(3-chlorophenyl)-2-oxoacetaldehyde oxime (220 mg, 1.2 mmol). The reaction was stirred for 1 month. The precipitate was filtrated off and washed with MeOH to give the title compound (111 mg, 35%) as a light yellow solid.

¹H-NMR (DMSO-d₆): δ 13.88 (s, 1H), 12.58 (s, 1H), 8.55 (d, 1H), 8.47 (s, 1H), 7.87-7.83 (m, 2H), 7.76 (td, 1H), 7.58 (dd, 1H), 7.52-7.47 (m, 2H), 2.67 (s, 3H).

MS (M⁺+H) m/z=317.

### Example 128

### N′-(2-(Ethoxyimino)-1-(3-chlorophenyl)ethylidene)benzohydrazide

To a solution of N′-(1-(3-chlorophenyl)-2-(hydroxyimino)ethylidene)-benzohydrazide (301 mg, 1.0 mmol; see Example 14) in dry DMF (10 mL) at RT was added bromoethane (82 μL, 1.1 mmol) and potassium carbonate (124 mg, 0.9 mmol). The suspension was stirred for 10 min. EtOAc was added and the organic phase was washed with 3 portions of NaOH (2M) and 2 portions of CaCl₂ (sat.), water and NaCl (sat.). The organic phase was dried (Na₂SO₄) and the solvent removed in vacuo. The residue was dissolved in EtOH and water was added resulting in precipitation, which was separated to give the title compound (27 mg, 8%) as a light yellow solid.

¹H-NMR (DMSO-d₆): δ 12.79 (s, 1H), 8.63 (s, 1H), 7.89 (d, 2H), 7.82 (s, 1H), 7.74 (d, 1H), 7.66 (t, 1H), 7.58 (t, 2H), 7.53-7.47 (m, 2H), 4.34 (q, 2H), 1.31 (t, 3H).

MS (M⁺+H) m/z=330.

### Example 129

### 5-Methylpyrazine-2-carboxylic acid (1-(3-chlorophenyl)-2-hydroxyimino-ethylidene)hydrazide

General procedure Y was employed using 5-methylpyrazine-2-carboxylic acid hydrazide (134 mg, 0.88 mmol) and 2-(3-chlorophenyl)-2-oxoacetaldehyde oxime (184 mg, 1 mmol)). The reaction was stirred for 1 month. The precipitate was separated and recrystallised (from EtOH/H₂O) to give the title compound (58 mg, 20%) as a brown solid.

¹H-NMR (DMSO-d₆): δ 13.83 (s, 1H), 12.73 (s, 1H), 9.17 (s, 1H), 8.62 (s, 1H), 8.48 (s, 1H), 7.86 (bs, 1H), 7.77 (d, 1H), 7.55-7.48 (m, 2H), 2.65 (s, 3H).

MS (M⁺+H) m/z=318.

### Example 130

### Benzoic acid (2-hydroxyimino-1-phenylethylidene)hydrazide

The title compound was prepared in accordance with the procedures described herein.

### Example 131

Title compounds of the Examples were tested in the biological test described above and were found to exhibit at least 50% inhibition of 15-lipoxygenase at a concentration of 10 μM or below.

1. A use of a compound of formula I, 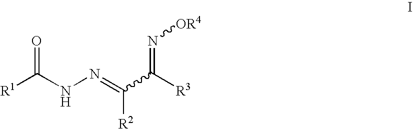
wherein
the squiggly bonds represent optional E or Z geometry;
R¹ and R² independently represent an aryl group or a heteroaryl group, both of which groups are optionally substituted by one or more substituents selected from:
X¹, C₁₋₈ alkyl, an aryl group and a heterocyclic group:—
(A) which C₁₋₈ alkyl group is itself optionally substituted by one or more Z substituents; and
(B) which C₁₋₈ alkyl, aryl and heterocyclic groups may themselves be substituted by one or more substituents selected from X¹, C₁₋₈ alkyl (which latter group may be further substituted by one or more substituents selected from X¹, C₁₋₈ alkyl, an aryl group, a heterocyclic group and Z), an aryl group and a heterocyclic group (and which latter two groups may be further substituted by one or more substituents selected from X¹, C₁₋₈ alkyl, an aryl group and a heterocyclic group), in which:—
X¹ represents, on each occasion when used above, halo, cyano, —N₃, —NO₂, —ONO₂ or -A¹-R⁵, wherein:
A¹ represents a spacer group selected from —C(Z)A²-, —N(R⁶)A³-, —OA⁴-, —S— or —S(O)ₙA⁵-, in which:
A² represents a single bond, —O—, —S—, —N(R⁶)A⁶- or —C(Z)-;
A³ represents A⁶, —C(Z)N(R⁶)C(Z)N(R⁶)—, —C(Z)N(R⁶)C(Z)O—, —C(Z)N(R⁶)S(O)ₙN(R⁶)—, —C(Z)S—, —S(O)ₙ—, —S(O)ₙN(R⁶)C(Z)N(R⁶)—, —S(O)ₙN(R⁶)C(Z)O— or —S(O)ₙN(R⁶)S(O)ₙN(R⁶)—;
A⁴ represents A⁶ or —S(O)ₙ—;
A⁵ represents a single bond, —N(R⁶)— or —O—;
A⁶ represents, on each occasion when used above, a single bond, —C(Z)-, —C(Z)O—, —C(Z)N(R⁶)—, —S(O)ₙN(R⁶)— or —S(O)ₙO—; and
Z represents, on each occasion when used above, a substituent connected by a double bond, which is selected from ═O, ═S, ═NR⁵, ═NN(R⁵)(R⁶), ═NOR⁵, ═NS(O)₂N(R⁵)(R⁶), ═NCN, ═C(H)NO₂ and ═C(R⁵)(R⁶);
R⁵ and R⁶ independently represent, on each occasion when used above,
(a) hydrogen;
(b) C₁₋₈ alkyl, optionally substituted by one or more substituents selected from X², Q, C₁₋₈ alkyl (optionally substituted by one or more substituents selected from X², C₁₋₈ alkyl, an aryl group, a heterocyclic group and Q), an aryl group and a heterocyclic group (which latter two groups are optionally substituted by one or more substituents selected from X², C₁₋₈ alkyl, an aryl group and a heterocyclic group); or
(c) an aryl group or a heterocyclic group, both of which are optionally substituted by one or more substituents selected from X², C₁₋₈ alkyl (optionally substituted by one or more substituents selected from X², C₁₋₈ alkyl, an aryl group, a heterocyclic group and Q), an aryl group and a heterocyclic group (which latter two groups are optionally substituted by one or more substituents selected from X², C₁₋₈ alkyl, an aryl group and a heterocyclic group); or
R⁵ and R⁶ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X², C₁₋₈ alkyl, an aryl group, a heterocyclic group (which latter three groups are optionally substituted as described in (b) and (c) above, respectively) and, provided that the ring that R⁵ and R⁶ may together be part of is not aromatic in character, Q;
X² represents, on each occasion when used above, halo, cyano, —N₃, —NO₂, —ONO₂ or -A⁷-R⁷, wherein:
A⁷ represents a spacer group selected from —C(Q)A⁸-, —N(R⁸)A⁹-, —OA¹⁰-, —S— or —S(O)ₙA¹¹-, in which:
A⁸ represents a single bond, —O—, —S—, —N(R⁸)— or —C(Q)-;
A⁹ represents A¹², —C(Q)N(R⁸)C(Q)N(R⁸)—, —C(Q)N(R⁸)C(Q)O—, —C(Q)N(R⁸)S(O)ₙN(R⁸)—, —C(Q)S—, —S(O)ₙ—, N(R⁸)C(Q)N(R⁸)—, —S(O)ₙN(R⁸)C(Q)N(R⁸)—, —S(O)ₙN(R⁸)C(Q)O— or —S(O)ₙN(R⁸)S(O)ₙN(R⁸)—;
A¹⁰ represents A¹² or —S(O)ₙ—;
A¹¹ represents a single bond, —N(R⁸)— or —O—;
A¹² represents, on each occasion when used above, a single bond, —C(Q)-, —C(Q)O—, —C(Q)N(R⁸)—, —S(O)ₙN(R⁸)— or —S(O)ₙO—;
Q represents, on each occasion when used above, a substituent connected by a double bond, which is selected from ═O, ═S, ═NR⁷, ═NN(R⁷)(R⁸), ═NOR⁷, ═NS(O)₂N(R⁷)(R⁸), ═NCN, ═C(H)NO₂ and ═C(R⁷)(R⁸);
R⁷ and R⁸ independently represent, on each occasion when used herein,
(i) hydrogen;
(ii) an aryl group or a heterocyclic group, both of which may be substituted by one or more substituents selected from X³, C₁₋₈ alkyl, an aryl group and a heterocyclic group (and which latter three groups are themselves optionally substituted by one or more substituents selected from halo, hydroxy,
—R⁹, —OR⁹ and, provided that the group is not aromatic in nature, ═O); or
(iii) C₁₋₈ alkyl, optionally substituted by one or more substituents selected from X³ and W; or
R⁷ and R⁸ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X³, C₁₋₈ alkyl, an aryl group, a heterocyclic group and, provided that the ring that R⁷ and R⁸ may together be part of is not aromatic in character, W;
X³ represents, on each occasion when used above, halo, cyano, —N₃, —NO₂, —ONO₂ or -A¹³-R¹⁰, wherein:
A¹³ represents a spacer group selected from —C(W)A¹⁴-, —N(R¹¹)A¹⁵-, —OA¹⁶-, —S— or —S(O)ₙA¹⁷, in which:
A¹⁴ represents a single bond, —O—, —S—, —N(R¹¹)— or —C(W)—,
A¹⁵ represents A¹⁸, —C(W)N(R¹¹)C(W)N(R¹¹)—, —C(W)N(R¹¹)C(W)O—, —C(W)N(R¹¹)S(O)ₙN(R¹¹)—, —C(W)S—, —S(O)ₙ—, —S(O)ₙN(R¹¹)C(W)N(R¹¹)—, —S(O)ₙN(R¹¹)C(W)O— or —S(O)ₙN(R¹¹)S(O)ₙN(R¹¹)—;
A¹⁶ represents A¹⁸ or —S(O)ₙ—;
A¹⁷ represents a single bond, —N(R¹¹)— or —O—;
A¹⁸ represents, on each occasion when used above, a single bond, —C(W)—, —C(W)O—, —C(W)N(R¹¹)—, —S(O)ₙN(R¹¹)— or —S(O)ₙO—
R⁹ represents, on each occasion when used above, C₁₋₆ alkyl optionally substituted by one or more fluoro atoms;
W represents, on each occasion when used above, a substituent connected by a double bond, which is selected from ═O, ═S, ═NR¹⁰, ═NN(R¹⁰)(R¹¹), ═NOR¹⁰, ═NS(O)₂N(R¹⁰)(R¹¹), ═NCN, ═C(H)NO₂ and ═C(R¹⁰)(R¹¹);
R¹⁰ and R¹¹ independently represent, on each occasion when used above:
(1) hydrogen;
(2) an aryl group or a heterocyclic group, both of which may be substituted by one or more substituents selected from X⁴, C₁₋₈alkyl, methylenedioxy, difluoromethylenedioxy and dimethylmethylenedioxy; or
(3) C₁₋₈ alkyl, optionally substituted by one or more substituents selected from X⁴, ═O, ═S, ═NR¹², ═NN(R¹²)(R¹³), ═NOR¹², ═NS(O)₂N(R¹²)(R¹³), ═NCN, ═C(H)NO₂ and ═C(R¹²)(R¹³); or
R¹⁰ and R¹¹ may, when present on the same atom or on adjacent atoms, taken together with those atoms form a 5- to 8-membered ring optionally containing 1 to 3 heteroatoms and/or 1 to 3 double bonds, which ring is itself optionally substituted by one or more substituents selected from X⁴ and, provided that the ring that R¹⁰ and R¹¹ may together be part of is not aromatic in character, ═O, ═S, ═NR¹², ═NN(R¹²)(R¹³), ═NOR¹², ═NS(O)₂N(R¹²)(R¹³), ═NCN, ═C(H)NO₂ and ═C(R¹²)(R¹³);
X⁴ represents, on each occasion when used above, halo, cyano, —N₃, —NO₂, —ONO₂ or -A¹⁹-R¹², wherein:
A¹⁹ represents a spacer group selected from —C(O)A²⁰-, —N(R¹³)A²¹-, —OA²²-, —S— or —S(O)ₙA²³-, in which:
A²⁰ represents a single bond, —O—, —S—, —N(R¹³)— or —C(O)—;
A²¹ represents A²⁴, —C(O)N(R¹³)C(O)N(R¹³)—, —C(O)N(R¹³)C(O)O—, —C(O)N(R¹³)S(O)ₙN(R¹³)—, —C(O)S—, —S(O)ₙ—, —S(O)ₙN(R¹³)C(O)N(R¹³)—, —S(O)ₙN(R¹³)C(O)O— or —S(O)ₙN(R¹³)S(O)ₙN(R¹³)—;
A²² represents A²⁴ or —S(O)ₙ—,
A²³ represents a single bond, —N(R¹³)— or —O—;
A²⁴ represents, on each occasion when used above, a single bond, —C(O)—, —C(O)O—, —C(O)N(R¹³)—, —S(O)ₙN(R¹³)— or —S(O)ₙO—;
R¹² and R¹³ independently represent, on each occasion when used above:
(A) hydrogen; or
(B) C₁₋₆ alkyl, optionally substituted by one or more substituents selected from halo, —N(R¹⁴)R¹⁵, —OR¹⁵ and ═O;
n represents, on each occasion when used above, 1 or 2;
R³ and R⁴ independently represent H or C₁₋₆ alkyl optionally substituted by one or more substituents selected from halo, C₁₋₆ alkyl, cyano, —NO₂, —ONO₂, —N(R¹⁴)R¹⁵, —OR¹⁵, ═O, aryl and heteroaryl; and
R¹⁴ and R¹⁵ independently represent, on each occasion when used above, H or C₁₋₄ alkyl,
or a pharmaceutically acceptable salt thereof;
for the manufacture of a medicament for the treatment of a disease in which inhibition of the activity of a lipoxygenase, and particularly 15-lipoxygenase, is desired and/or required.

2. A use as claimed in claim 1, wherein R¹ represents a phenyl group or a heteroaryl group, both of which are optionally substituted by one or more substituents selected from X¹, aryl and C₁₋₆ alkyl, which alkyl group is optionally substituted by one or more groups selected from X¹.

3. A use as claimed in claim 1, wherein R² represents a phenyl group or a heteroaryl group, both of which are optionally substituted by one or more substituents selected from X¹, a heterocyclic group and C₁₋₃ alkyl, which alkyl group is optionally substituted by X¹.

4. A use as claimed in claim 1, wherein X¹ represents halo, —NO₂, -A¹-R⁵ or cyano.

5. A use as claimed in claim 1, wherein A¹ represents —N(R⁶)A³-, —OA⁴- or —S(O)ₙA⁵-.

6. A use as claimed in claim 1, wherein A² represents a single bond, —O— or —N(R⁶)A⁶-.

7. A use as claimed in claim 1, wherein A³ represents A⁶ or —S(O)ₙ—.

8. A use as claimed in claim 1, wherein A⁴ represents A⁶.

9. A use as claimed in claim 1, wherein A⁵ represents —N(R⁶)—.

10. A use as claimed in claim 1, wherein A⁶ represents a single bond.

11. A use as claimed in claim 1, wherein n represents 2.

12. A use as claimed in claim 1, wherein R⁵ represents H, C₁₋₃ alkyl or phenyl.

13. A use as claimed in claim 1, wherein R⁶ represents H or C₁₋₃ alkyl.

14. A use as claimed in claim 1, wherein R³ represents H or C₁₋₃ alkyl.

15. A use as claimed in claim 1, wherein R⁴ represents H or C₁₋₃ alkyl, which alkyl group is optionally substituted by one or more substituents selected from phenyl and —OR¹⁵.

16. A use as claimed in claim 1, wherein R¹⁵ represents C₁₋₂ alkyl.

17. A use as claimed in claim 1, wherein R¹ and/or R² represent an optionally substituted pyrrolidinyl, piperidinyl, oxindolyl, phenyl, naphthyl, pyrrolyl, furanyl, thienyl, pyrazolyl, imidazolyl, oxazolyl, isoxazolyl, thiazolyl, pyridyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, benzodioxanyl, tetrazolyl, benzothiazolyl and/or indazolyl group.

18. A use as claimed in claim 17, wherein R¹ and/or R² represent an optionally substituted pyrrolidinyl, piperidinyl, oxindolyl, phenyl, naphthyl, pyrrolyl, furanyl, thienyl, pyrazolyl, imidazolyl, oxazolyl, isoxazolyl, thiazolyl, pyridyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl and/or benzodioxanyl group.

19. A use as claimed in claim 17, wherein the optional substituents are selected from halo, —NO₂, cyano, C₁₋₆ alkyl (which alkyl group may be linear, branched, cyclic, part-cyclic, unsaturated and/or optionally substituted with one or more fluoro group), phenyl, pyrrolidinyl, piperidinyl, piperazinyl, tetrahydrofuranyl, tetrahydropyranyl, morpholinyl, —OR¹⁸, —N(R⁸)R¹⁹, —C(O)R¹⁸, —C(O)OR¹⁸—C(O)N(R¹⁸), R¹⁹, —S(O)ₘR²⁰, —S(O)₂N(R¹⁸)R¹⁹ and/or —N(R¹⁸)S(O)₂R²⁰, wherein R¹⁸ and R¹⁹ independently represent H, phenyl or C₁₋₆ alkyl, R²⁰ represents C₁₋₄ alkyl and m represents 0, 1 or 2.

20. A use as claimed in claim 19, wherein the substituents are selected from fluoro, chloro, bromo, cyano, hydroxyl, amino, —NO₂, C₁₋₄ alkyl, C₁₋₄ alkoxy, phenyl, phenoxy, trifluoromethyl, —N(H)SO₂CH₃, —SO₂NH₂, —SO₂N(CH₃)₂, —SO₂N(H)CH₃, —N(CH₃)₂ and morpholinyl.

21. A use as claimed in claim 20, wherein the substituents are selected from fluoro, chloro, bromo, cyano, hydroxyl, amino, —NO₂, C₁₋₄ alkyl, C₁₋₄ alkoxy, phenyl, phenoxy, trifluoromethyl, —N(H)SO₂CH₃, —SO₂NH₂ and —SO₂N(CH₃)₂.

22. A use as claimed in claim 1, wherein R¹ represents thienyl; pyrazolyl, which pyrazolyl group is substituted by one or more methyl groups or is unsubstituted; pyridyl, which pyridyl group is substituted by one or more substituents selected from bromo, chloro, methyl and hydroxyl or is unsubstituted; or phenyl, which phenyl group is optionally substituted by one or more substituents selected from methyl, t-butyl, methoxy, fluoro, chloro, bromo, trifluoromethyl, phenyl, hydroxyl, amino, —NO₂, —SO₂NH₂, —SO₂N(CH₃)₂, —SO₂N(H)CH₃ and —N(CH₃)₂.

23. A use as claimed in claim 22, wherein R¹ represents thienyl; unsubstituted pyrazolyl; unsubstituted pyridyl; or phenyl optionally substituted by one or more substituents selected from methyl, t-butyl, methoxy, fluoro, chloro, bromo, trifluoromethyl, phenyl, hydroxyl, amino, —NO₂, —SO₂NR₂ and —SO₂N(CH₃)₂.

24. A use as claimed in claim 1, wherein R² represents 2-pyridyl, 4-pyridyl, 2-quinoxalinyl, or phenyl, which phenyl group is optionally substituted by one or more substituents selected from methyl, phenoxy, —N(H)SO₂CH₃, methoxy, fluoro, chloro, bromo, trifluoromethyl, hydroxyl, —NO₂, —SO₂NH₂, —SO₂N(CH₃)₂, cyano, morpholinyl, —N(CH₃)₂ and ethyl.

25. A use as claimed in claim 24, wherein R² represents 2-pyridyl or phenyl, which phenyl group is optionally substituted by one or more substituents selected from methyl, phenoxy, —N(H)SO₂CH₃, methoxy, fluoro, chloro, bromo, trifluoromethyl, hydroxyl, —NO₂, —SO₂NH₂ and —SO₂N(CH₃)₂.

26. A compound of formula I as defined in claim 1, or a pharmaceutically acceptable salt thereof, for use as a pharmaceutical, provided that, when R⁴ represents H and:
(A) R³ represents H and:
(I) R² represents phenyl, then R¹ does not represent 2-furanyl, 4-pyridyl, 3-(5-methylisooxazolyl), phenyl, or 3-nitro-, 2-hydroxy-, 2-hydroxy-3-methyl-, 4-(phenylthio)-, 2-hydroxy-5-methyl- or 4-hydroxyphenyl;
(II) R² represents 4-chlorophenyl, then R¹ does not represent 2-furanyl, 4-pyridyl, phenyl, or 2-hydroxy-5-methyl-, 4-hydroxy-, 2-hydroxy-3-methyl- or 2-hydroxyphenyl;
(III) R² represents 4-methylphenyl, then R¹ does not represent 4-pyridyl, phenyl, or 3-nitro-, 2-hydroxy-5-methyl-, 4-hydroxy-, 2-hydroxy- or 4-(phenylthio)phenyl; or
(IV) R² represents 2-furanyl or 2-benzofuranyl, then R¹ does not represent 4-pyridyl or 3-(5-methylisooxazolyl); and
(B) R³ represents methyl and:
(1) R² represents phenyl, then R¹ does not represent N-(4-bromophenyl)-2-amino-, N-(2-methoxyphenyl)-2-amino-, N-(2-ethoxyphenyl)-2-amino-, N-(3-chlorophenyl)-2-amino-, N-(4-methylphenyl)-2-amino-, N-(3-methylphenyl)-2-amino-, N-(2-methylphenyl)-2-amino- or N-(phenyl)-2-aminophenyl; or
(2) R² represents 4-chlorophenyl, then R¹ does not represent 4-pyridyl, phenyl, or 3-nitro-, 2-hydroxy-5-methyl-, 4-hydroxy-, 2-hydroxy- or 2-hydroxy-3-methylphenyl.

27. A compound as claimed in claim 26, with the additional provisos that, when R⁴ represents H, R³ represents H and R² represents phenyl or 4-methylphenyl, then R¹ does not represent 4-(thienyl)phenyl.

28. A pharmaceutical formulation including a compound as defined in claim 26, or a pharmaceutically acceptable salt thereof, in admixture with a pharmaceutically acceptable adjuvant, diluent or carrier.

29. A compound as defined in claim 26, or a pharmaceutically-acceptable salt thereof, with the additional provisos that, when R⁴ represents H, R² represents phenyl and:
(a) R³ represents H, then R¹ does not represent 2-pyridyl, or 3-bromo-, 3,4-dimethoxy- or 2-hydroxy-5-bromophenyl; and
(b) R³ represents methyl, then R¹ does not represent 4-methoxyphenyl.

30. A use as claimed in claim 1 wherein the lipoxygenase is 15-lipoxygenase.

31. A use as claimed in claim 1, wherein the disease is inflammation and/or has an inflammatory component.

32. A use as claimed in claim 31 wherein the inflammatory disease is asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease or another malignancy.

33. A method of treatment of a disease in which inhibition of the activity of a lipoxygenase is desired and/or required, which method comprises administration of a therapeutically effective amount of a compound of formula I as defined in claim 1, or a pharmaceutically-acceptable salt thereof, to a patient suffering from, or susceptible to, such a condition.

34. A combination product comprising:
(A) a compound of formula I, as defined in claim 1, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

35. A pharmaceutical formulation including a compound of formula I as defined in claim 1, or a pharmaceutically-acceptable salt thereof, another therapeutic agent that is useful in the treatment of inflammation, and a pharmaceutically-acceptable adjuvant, diluent or carrier.

36. A kit of parts comprising components:
(a) a pharmaceutical formulation including a compound of formula I as defined in claim 1, or a pharmaceutically-acceptable salt thereof in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

37. A process for the preparation of a compound as defined in claim 29, which comprises:
(i) reaction of a compound of formula II, 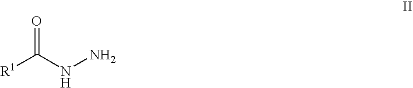
wherein R¹ is as defined in claim 1, or an acid addition salt thereof, with a compound of formula III, 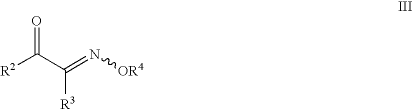
wherein the squiggly bond, R², R³ and R⁴ are as defined in claim 1;
(ii) reaction of a compound of formula IV, 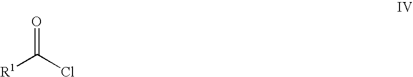
wherein R¹ is as defined in claim I with a compound of formula V, 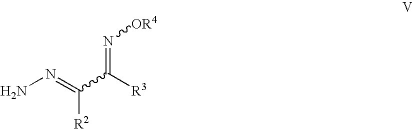
wherein the squiggly bonds, R², R³ and R⁴ are as defined in claim I;
(iii) reaction of a compound of formula VI, 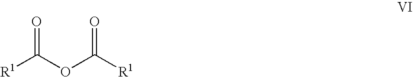
wherein R¹ is as defined in claim 1 with a compound of formula V as defined above;
(iv) ring opening of a compound of formula VII, 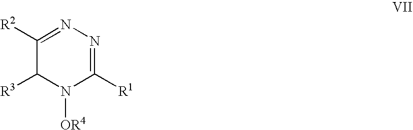
wherein R¹, R², R³ and R⁴ are as defined in claim 1;
(v) reaction of a compound of formula VIII, 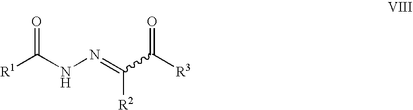
wherein the squiggly bond, R¹, R² and R³ are as defined in claim 1 with a compound of formula IX,
R⁴ONH₂ IX
wherein R⁴ is as defined in claim 1, or an acid addition salt thereof;
(vi) for compounds of formula I in which R⁴ represents optionally substituted C₁₋₆ alkyl, reaction of a corresponding compound of formula I in which R⁴ represents H with a compound of formula X,
R^{4a}L¹ X
wherein L¹ is a suitable leaving group and R^{4a} is C₁₋₆ alkyl optionally substituted by one or more substituents selected from halo, C₁₋₆ alkyl, cyano, —NO₂, —ONO₂, —N(R¹⁴)R¹⁵, —OR¹⁵, ═O, aryl or heteroaryl; or
(vii) reaction of a compound of formula X¹,
R¹L² XI
wherein L² is a suitable leaving group and R¹ is as defined in claim 1 with a compound of formula V as defined above in the presence of carbon monoxide or another suitable CO source.
